(19) United States
(12) Patent Application Publication
(10) Pub. No.: US 2024/0049953 A1
(43) Pub. Date: Feb. 15, 2024

(54) ENDOSCOPE FLUID CONTROL SYSTEM, FLUID CONTROL APPARATUS FOR ENDOSCOPE, CONTROL METHOD OF ENDOSCOPE FLUID CONTROL SYSTEM, AND CONTROL METHOD OF FLUID CONTROL APPARATUS FOR ENDOSCOPE

(71) Applicant: OLYMPUS MEDICAL SYSTEMS CORP., Tokyo (JP)

(72) Inventors: Nagahide SAKAI, Tokyo (JP); Satoshi HORIE, Tokyo (JP)

(73) Assignee: OLYMPUS MEDICAL SYSTEMS CORP., Tokyo (JP)

(21) Appl. No.: 18/211,677

(22) Filed: Jun. 20, 2023

Related U.S. Application Data
(60) Provisional application No. 63/356,055, filed on Jun. 28, 2022.

(51) Int. Cl. A61B 1/015; A61B 1/00; G16H 40/60

(52) U.S. Cl. CPC A61B 1/015; A61B 1/00006; G16H 40/60

(57) ABSTRACT

An endoscope fluid control system, includes: a water suction pump and a water suction pressure gauge connected to a water suction conduit; a memory configured to record correlation data for each type of endoscope, the correlation data indicating a correlation between a pressure value indicated by the water suction pressure gauge and an output value of the water suction pump; and a processor configured to: specify correlation data corresponding to a type; and monitor an operation of the water suction pump based on the specified correlation data.

### RELATED APPLICATION DATA

This application is based on and claims priority under 35 U.S.C. § 119 to U.S. Provisional Application No. 63/356,055 filed on Jun. 28, 2022, the entire contents of which are incorporated herein by reference.

### BACKGROUND OF THE INVENTION

### 1. Field of the Invention

The present invention relates to an endoscope fluid control system that generates a perfusion by performing water feeding and water suction, a fluid control apparatus for endoscope, a control method of the endoscope fluid control system, and a control method of the fluid control apparatus for endoscope.

### 2. Description of the Related Art

An endoscope fluid control system has been proposed which, with respect to a perfusion object with a dead-end structure such as inside a kidney, fragments a stone inside the perfusion object into crushed stone pieces with a laser, executes water feeding and water suction in a combined manner to generate a perfusion inside the perfusion object, and collects the stone from inside the perfusion object.

For example, International Publication No. 2019/176131 describes an endoscope system including a channel for an optical fiber, a channel for water feeding, and a channel for water suction.

The collection of a stone by a perfusion is performed in a state where a space inside a perfusion object such as an organ is secured. However, pressure (internal pressure) inside the perfusion object changes in accordance with a balance between water feeding and water suction. Therefore, the space inside the perfusion object must be secured by subjecting a liquid fed into the perfusion object to some degree of pressure and adequately maintaining the pressure.

Adequately maintaining internal pressure requires measuring the internal pressure. To this end, a pressure sensor or the like is arranged in a distal end portion of an insertion portion of an endoscope and a pressure sensor is arranged inside the perfusion object to measure internal pressure.

In this case, when cases where a pressure value measured by the pressure sensor is inaccurate are taken into consideration, a conceivable method involves arranging a plurality of pressure sensors inside the perfusion object to ensure accuracy of measured pressure values. For example, when two pressure sensors are arranged inside the perfusion object, a determination that an abnormality has occurred in any of the pressure sensors can be made when a difference equal to or larger than a threshold arises between pressure values measured by the respective pressure sensors.

### SUMMARY OF THE INVENTION

An endoscope fluid control system according to an aspect of the present invention includes: an endoscope including a first water suction conduit; a second water suction conduit detachably connected to the first water suction conduit; a pump connected to the second water suction conduit; a pressure gauge installed on the second water suction conduit; a memory configured to record correlation data which indicates a correlation between a pressure value indicated by the pressure gauge and an output value of the pump and which is set for each type of the endoscope; and a processor, wherein the processor is configured to: specify correlation data corresponding to a type of the endoscope the first water suction conduit of which is connected to the second water suction conduit; and monitor an operation of the pump based on the specified correlation data.

A control method of an endoscope fluid control system according to an aspect of the present invention includes, in a state where a first water suction conduit included in an endoscope and a second water suction conduit connected to a pump are connected to each other, specifying, from correlation data which indicates a correlation between a pressure value indicated by a pressure gauge installed on the second water suction conduit and an output value of the pump, which is set for each type of the endoscope, and which is recorded in a memory, correlation data corresponding to a type of the endoscope the first water suction conduit of which is connected to the second water suction conduit; and monitoring an operation of the pump based on the specified correlation data.

A fluid control apparatus for endoscope according to an aspect of the present invention includes: a second water suction conduit detachably connected to a first water suction conduit included in an endoscope; a pump connected to the second water suction conduit; a pressure gauge installed on the second water suction conduit; a memory configured to record correlation data which indicates a correlation between a pressure value indicated by the pressure gauge and an output value of the pump and which is set for each type of the endoscope; and a processor, wherein the processor is configured to: specify correlation data corresponding to a type of the endoscope the first water suction conduit of which is connected to the second water suction conduit; and monitor an operation of the pump based on the specified correlation data.

A fluid control apparatus for endoscope according to an aspect of the present invention includes: a second water suction conduit detachably connected to a first water suction conduit included in an endoscope; a pump connected to the second water suction conduit; a pressure gauge installed on the second water suction conduit; a memory configured to record first minimum correlation data which indicates a correlation between a pressure value indicated by the pressure gauge and an output value of the pump and which is constructed based on a first pressure value indicated by the pressure gauge and an output value of the pump corresponding to the first pressure value; and a processor, wherein the processor is configured to: construct second minimum correlation data based on a second pressure value measured by the pressure gauge and an output value of the pump corresponding to the second pressure value when the pump is operated by connecting the first water suction conduit to the second water suction conduit; construct correlation data based on a combination of the first minimum correlation data and the second minimum correlation data; and monitor an operation of the pump based on the constructed correlation data.

A control method of a fluid control apparatus for endoscope according to an aspect of the present invention includes: operating a pump in a state where a first water suction conduit included in an endoscope and a second water suction conduit connected to the pump are connected to each other; reading, from a memory, first minimum correlation data which indicates a correlation between a pressure value indicated by a pressure gauge installed on the second water suction conduit and an output value of the pump and which is constructed based on a first pressure value indicated by the pressure gauge and an output value of the pump corresponding to the first pressure value; constructing second minimum correlation data based on a second pressure value measured by the pressure gauge and an output value of the pump corresponding to the second pressure value; constructing correlation data based on a combination of the first minimum correlation data and the second minimum correlation data; and monitoring an operation of the pump based on the constructed correlation data.

### BRIEF DESCRIPTION OF THE DRAWINGS

FIG. 1 is a diagram showing a configuration example of an endoscope fluid control system according to a first embodiment of the present invention;

FIG. 2 is a diagram showing a configuration example related to a fluid path of the endoscope fluid control system according to the first embodiment of the present invention;

FIG. 3 is a perspective view showing a configuration of a distal end portion of an insertion portion of an endoscope according to the first embodiment of the present invention;

FIG. 4 is a block diagram showing a functional configuration of a controller according to the first embodiment of the present invention;

FIG. 5 is a diagram schematically showing a flow of a fluid of a water suction system in the endoscope fluid control system in a state where a valve is closed in the first embodiment of the present invention;

FIG. 6 is a diagram schematically showing a flow of a fluid of the water suction system in the endoscope fluid control system in a state where the valve is open in the first embodiment of the present invention;

FIG. 7 is a graph showing a change over time of a pressure value Pi measured by an internal pressure sensor and a pressure value P2 measured by a water suction pressure gauge when performing water feeding and water suction to a perfusion object in the first embodiment of the present invention;

FIG. 8 is a bar chart showing a relative pressure change amount of the pressure value Pi and the pressure value P2 in a range in a stable state when an operation condition involving setting a drive voltage of a water feed pump to 2 V is changed to an operation condition involving other drive voltages in the graph shown in FIG. 7 according to the first embodiment of the present invention;

FIG. 9 is a chart in which graphs indicating a relationship between a drive voltage of a pump and a pressure value measured by a pressure gauge in a water feed system and the water suction system are arranged in the first embodiment of the present invention;

FIG. 10 is a chart in which graphs indicating a normal range in a relationship between a drive voltage of a pump and a pressure value measured by a pressure gauge in the water feed system and the water suction system are arranged in the first embodiment of the present invention;

FIG. 11 is a flowchart showing pressure reference creation processing by the endoscope fluid control system according to the first embodiment of the present invention;

FIG. 12 is a flowchart showing water feed abnormality detection processing according to the first embodiment of the present invention;

FIG. 13 is a flowchart showing water suction abnormality detection processing according to the first embodiment of the present invention;

FIG. 14 is a flowchart showing clogging detection processing according to the first embodiment of the present invention;

FIG. 15 is a block diagram showing a configuration example of an internal pressure sensor abnormality detecting unit according to the first embodiment of the present invention;

FIG. 16 is a flowchart showing internal pressure sensor abnormality detection processing according to the first embodiment of the present invention;

FIG. 17 is a graph showing a relationship between a flow rate of a water suction conduit and pressure loss in the first embodiment of the present invention;

FIG. 18 is a graph showing a change with elapsed time of a water suction flow rate and a pressure value measured by an internal pressure sensor according to the first embodiment of the present invention;

FIG. 19 is a flowchart showing processing of estimating pressure inside a perfusion object from a flow rate measured by a water suction flow rate meter in the first embodiment of the present invention;

FIG. 20 is a timing chart showing a first example of a method of stopping a water feed pump and a water suction pump according to the first embodiment of the present invention;

FIG. 21 is a timing chart showing a second example of a method of stopping a water feed pump and a water suction pump according to the first embodiment of the present invention;

FIG. 22 is a timing chart showing a third example of a method of stopping a water feed pump and a water suction pump according to the first embodiment of the present invention;

FIG. 23 is a timing chart showing a fourth example of a method of stopping a water feed pump and a water suction pump according to the first embodiment of the present invention;

FIG. 24 is a timing chart showing a fifth example of a method of stopping a water feed pump and a water suction pump according to the first embodiment of the present invention; and

FIG. 25 is a flowchart showing an example of internal pressure feedback control processing upon stopping a water feed pump and a water suction pump according to the first embodiment of the present invention.

### DETAILED DESCRIPTION OF THE PREFERRED EMBODIMENT

Hereinafter, an embodiment of the present invention will be described with reference to the drawings. However, it is to be understood that the present invention is not limited by the embodiment described below.

In the description of the drawings, same or corresponding elements are denoted by same reference signs when appropriate. In addition, it should be noted that drawings are schematic and a relationship among lengths of respective elements, a ratio among lengths of respective elements, a quantity of each element, and the like in a single drawing may differ from reality for the sake of brevity of description. Furthermore, even among a plurality of drawings, the drawings may include portions having a relationship or a ratio among lengths that differ from each other.

### First Embodiment

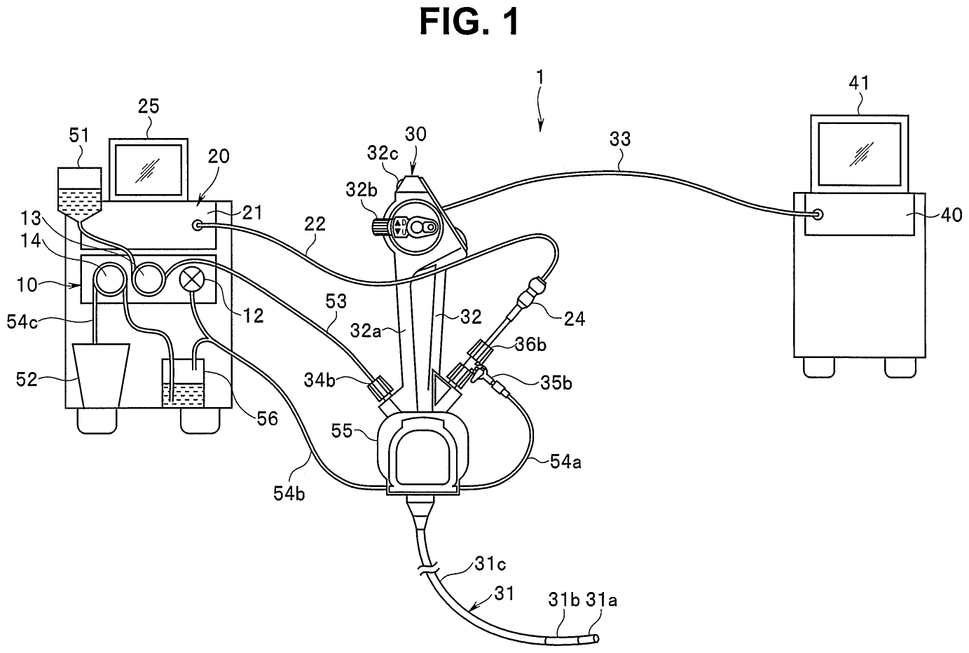

FIGS. 1 to 25 represent a first embodiment of the present invention. FIG. 1 is a diagram showing a configuration example of an endoscope fluid control system according to the first embodiment.

As shown in FIG. 1, an endoscope fluid control system 1 according to the present embodiment is configured as an endoscope system and includes a fluid control apparatus for endoscope 10, a laser system 20, an endoscope 30, and a video processor 40.

The endoscope 30 is a device for performing an observation and treatment of a subject. The endoscope 30 includes an elongated insertion portion 31 to be inserted into the subject, an operating unit 32 that is provided on a side of a proximal end of the insertion portion 31, and a universal cable 33 that is extended from the operating unit 32. Note that a living body such as a person/human being or an animal is assumed as the subject into which the insertion portion 31 is inserted. In addition, a specific example of a perfusion object 90 according to the endoscope fluid control system 1 is a kidney (a renal pelvis, a renal calyx, or the like), a ureter, a urinary bladder, a urethra, or the like.

The insertion portion 31 includes, in order from a distal end toward a proximal end, a distal end portion 31a, a bending portion 31b, and a tubular portion 31c.

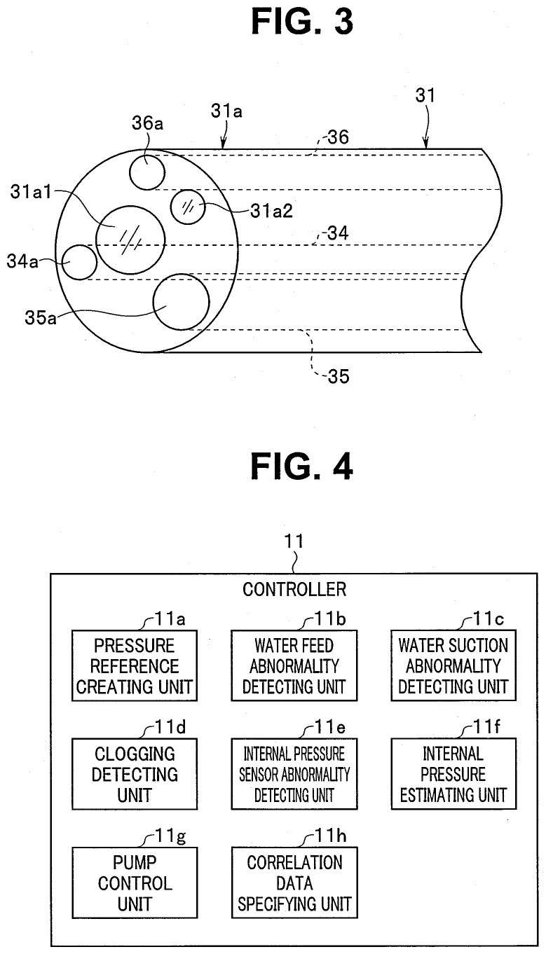

The distal end portion 31a includes an observing system and an illuminating system. The observing system forms an optical image of the subject using an objective optical system 31a1 (refer to FIG. 3) and photoelectrically converts the optical image with an image pickup device to generate an image pickup signal. The illuminating system transmits illuminating light with, for example, a light guide and irradiates the subject with the illuminating light from an illumination window 31a2 (refer to FIG. 3). A signal line connected to the image pickup device and the light guide are arranged inside the insertion portion 31, the operating unit 32, and the universal cable 33 and are connected to the video processor 40 that doubles as, for example, a light source apparatus.

FIG. 3 is a perspective view showing a configuration of the distal end portion 31a of the insertion portion 31 of the endoscope 30 according to the first embodiment.

The insertion portion 31 includes a water feed channel 34, a water suction channel 35, and a treatment instrument channel 36. The water feed channel 34 includes a channel opening 34a in the distal end portion 31a. The water suction channel 35 includes a channel opening 35a in the distal end portion 31a. The treatment instrument channel 36 includes a channel opening 36a in the distal end portion 31a.

The water feed channel 34 is configured to transport a liquid (fluid) such as normal saline and feed the liquid into the subject from the channel opening 34a.

The water suction channel 35 is configured to suction (perform water suction of) the liquid (fluid) inside the subject from the channel opening 35a together with, for example, fragmented stones. While the water suction channel 35 is also referred to as a suction channel, the name “water suction” will be used in the present specification in order to be consistent with the water feed channel 34.

A treatment instrument including a laser probe 22 is inserted into the treatment instrument channel 36. Note that a treatment instrument such as forceps may be inserted into the treatment instrument channel 36 instead of the laser probe 22.

While FIG. 3 shows an example in which the endoscope 30 includes the treatment instrument channel 36 separately from the water feed channel 34 and the water suction channel 35, the endoscope 30 is not limited to this configuration. For example, a configuration may be adopted in which the water suction channel 35 doubles as a treatment instrument channel and the laser probe 22 is to be inserted into the water suction channel 35.

The bending portion 31b is provided on a side of a proximal end of the distal end portion 31a and is configured to be capable of bending in, for example, two directions or in four directions of upward, downward, leftward, and rightward. When the bending portion 31b is bent, a direction of the distal end portion 31a changes and a direction of observation by the observing system and an irradiation direction of illuminating light by the illuminating system change. In addition, the bending portion 31b is also bent in order to improve insertability of the insertion portion 31 in the subject.

The tubular portion 31c is a tubular part that couples a proximal end of the bending portion 31b and a distal end of the operating unit 32 to each other. The tubular portion 31c has a flexible form which deflects in accordance with, for example, a shape of the subject into which the tubular portion 31c is inserted.

The operating unit 32 is a part which is provided on a side of the proximal end of the insertion portion 31 and which is used to perform various operations with respect to the endoscope 30. For example, the operating unit 32 includes a grasping portion 32a, a bending operation lever 32b, an operation button 32c, a proximal end-side channel opening 34b of the water feed channel 34, a proximal end-side channel opening 36b of the treatment instrument channel 36, and a water suction tube connector 35b of the water suction channel 35. When the water suction channel 35 doubles as a treatment instrument channel, the channel opening 36b and the water suction tube connector 35b are provided in a T-tube.

The grasping portion 32a is a part by which a user grasps the endoscope 30 with a hand.

The bending operation lever 32b is an operation device for performing an operation of bending the bending portion 31b using, for example, a thumb of the hand grasping the grasping portion 32a.

For example, the operation button 32c includes a water feed button and a water suction button. The water feed button is an operation button for performing water feeding to a side of the distal end portion 31a via the water feed channel 34. The water suction button is an operation button for performing water suction from the side of the distal end portion 31a via the water suction channel 35. In addition, the plurality of operation buttons 32c may include, for example, a button switch for performing operations related to image pickup (such as a release operation).

The proximal end-side channel opening 34b of the water feed channel 34 is provided on a side surface on a side of a distal end of the grasping portion 32a. A water feed tube 53 is connected to the channel opening 34b. The water feed channel 34 and the water feed tube 53 constitute a water feed conduit.

The proximal end-side channel opening 36b of the treatment instrument channel 36 is provided on another side surface on the side of the distal end of the grasping portion 32a. The laser probe 22 is inserted using a protective tube 24 into the channel opening 36b of the treatment instrument channel 36. The protective tube 24 prevents an optical fiber included in the laser probe 22 from breaking.

The water suction channel 35 suctions a liquid inside the subject from the channel opening 35a together with fragmented stones. A first water suction tube 54a is detachably connected to the water suction tube connector 35b provided on the proximal end side of the water suction channel 35. The first water suction tube 54a, a second water suction tube 54b, and a third water suction tube 54c will be collectively referred to as a water suction tube 54 when appropriate. The water suction channel 35 (the first water suction conduit) and the water suction tube 54 (the second water suction conduit) constitute a water suction conduit. While the water suction tube 54 will also be referred to as a suction tube and the water suction conduit will also be referred to as a suction conduit, the name “water suction” will be used for a similar reason as the water suction channel 35.

For example, the universal cable 33 is extended from a side surface on a side of a proximal end of the operating unit 32 and is connected to the video processor 40.

The video processor 40 doubles as a light source apparatus and is configured to control the endoscope 30, process an image pickup signal acquired from the endoscope 30, and supply the endoscope 30 with illuminating light. The video processor 40 is configured to perform image processing on the image pickup signal and generate a displayable image signal. The image signal generated by the video processor 40 is outputted to a monitor 41 and an endoscopic image is displayed on the monitor 41. Alternatively, a light source apparatus may be configured separately from the video processor 40.

The laser system 20 is a stone fragmentation apparatus including a laser light source apparatus 21 and the laser probe 22. The laser light source apparatus 21 is configured to generate laser light (energy) for fragmenting a stone (urinary tract stone) on a urinary tract including the kidney, the ureter, the urinary bladder, and the urethra and to supply the laser probe 22 with the generated laser light. The laser probe 22 includes an optical fiber for transmitting laser light. A distal end of the laser probe 22 is extended out from the distal end-side channel opening 35a of the water suction channel 35 and laser light is generated by the laser light source apparatus 21. As a result, laser light transmitted by the optical fiber of the laser probe 22 is emitted to the stone inside the subject from a distal end of the optical fiber and the stone is fragmented and becomes crushed stone pieces.

While the laser system 20 is described as an example of a stone fragmentation apparatus in the present embodiment, the stone fragmentation apparatus is not limited thereto and may be any apparatus capable of fragmenting a stone. For example, when fragmenting a stone using ultrasound, an ultrasound probe and an ultrasound apparatus configured to supply the ultrasound probe with energy may be used as the stone fragmentation apparatus in place of the laser probe 22 and the laser light source apparatus 21.

The fluid control apparatus for endoscope 10 includes a valve 12, a water feed pump 13, and a water suction pump 14. A display apparatus 25 configured to display various types of information is connected to the fluid control apparatus for endoscope 10 and the laser system 20. In comparison to the monitor 41 described above configured to display endoscopic images, the display apparatus 25 is configured to display information and the like related to fluid control and laser irradiation.

Furthermore, a water feed source 51, a wastewater tank 52, the water feed tube 53, a primary strainer 55, and a secondary strainer 56 are provided on a fluid path of the endoscope fluid control system 1.

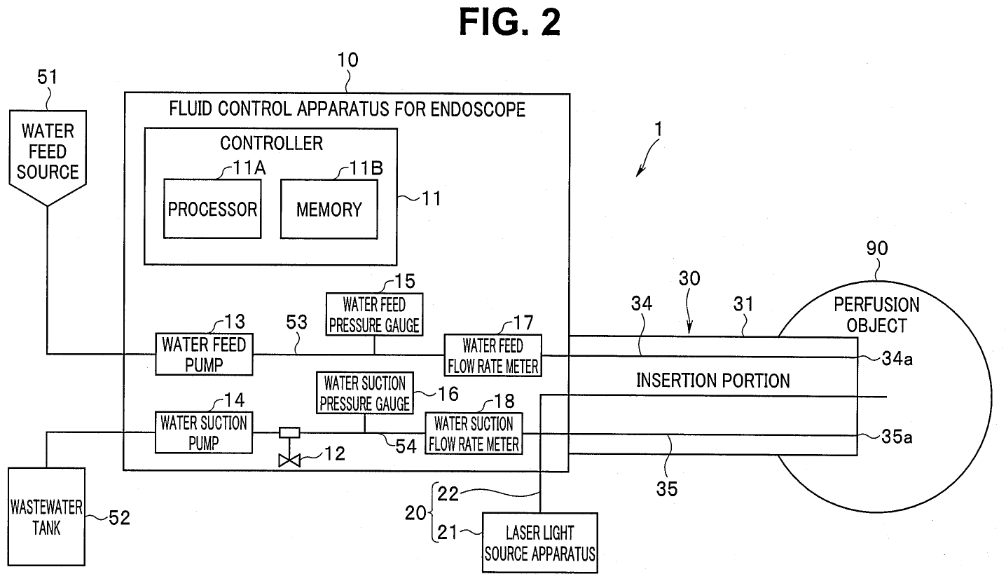

FIG. 2 is a diagram showing a configuration example related to the fluid path of the endoscope fluid control system 1 according to the first embodiment.

As shown in FIG. 2, the fluid control apparatus for endoscope 10 further includes a controller 11, a water feed pressure gauge 15, a water suction pressure gauge 16, a water feed flow rate meter 17, and a water suction flow rate meter 18.

The water feed source 51, the water feed tube 53, the water feed pump 13, the water feed pressure gauge 15, the water feed flow rate meter 17, and the water feed channel 34 constitute the water feed system. Note that arrangement sequences of the water feed pressure gauge 15 and the water feed flow rate meter 17 in the water feed system are not limited to the example shown in FIG. 2.

The water suction channel 35, the water suction tube 54, the water suction flow rate meter 18, the water suction pressure gauge 16, the water suction pump 14, the valve 12, and the wastewater tank 52 constitute the water suction system. Note that arrangement sequences of the water suction flow rate meter 18, the water suction pressure gauge 16, and the valve 12 in the water suction system are not limited to the examples shown in FIGS. 2, 5, and 6. The water feed system and the water suction system will be collectively referred to as a fluid system when appropriate.

The water feed source 51 is configured to store a liquid to be fed into the subject. The liquid stored in the water feed source 51 is, for example, normal saline as described above.

The water feed source 51 is connected to the water feed pump 13 by the water feed tube 53 and the water feed tube 53 on a distal end side relative to the water feed pump 13 is connected to the proximal end-side channel opening 34b of the water feed channel 34.

The water feed pressure gauge 15 is provided between, for example, the water feed pump 13 and the water feed flow rate meter 17 on the water feed tube 53. The water feed pressure gauge 15 includes a pressure sensor or the like and is configured to detect pressure of a fluid inside the water feed tube 53. A processor 11A of the controller 11 is configured to acquire pressure detected by the water feed pressure gauge 15.

The water feed pump 13 is configured to feed a liquid supplied from the water feed source 51 to the water feed tube 53 and the water feed channel 34 and to discharge the liquid into the perfusion object 90 of the subject from the channel opening 34a.

Alternatively, instead of performing water feeding via the water feed channel 34, a configuration may be adopted in which the water feed tube 53 is inserted into the water feed channel 34, the distal end of the water feed tube 53 is caused to protrude from a distal end surface of the distal end portion 31a of the insertion portion 31, and water feeding is performed from the distal end of the water feed tube 53.

When connected to the water suction tube connector 35b, the first water suction tube 54a communicates with the inside of the water suction channel 35. In addition, the first water suction tube 54a is connected to the primary strainer 55.

Alternatively, instead of performing water suction via the water suction channel 35, a configuration may be adopted in which the first water suction tube 54a is inserted into the water suction channel 35, the distal end of the first water suction tube 54a is caused to protrude from a distal end surface of the distal end portion 31a of the insertion portion 31, and water suction is performed from the distal end of the first water suction tube 54a.

Of the second water suction tube 54b, a first end is connected to the primary strainer 55 and another end is connected to the secondary strainer 56. In addition, the second water suction tube 54b has a branch portion that branches between the water suction flow rate meter 18 and the water suction pump 14 and the valve 12 is connected to an end part of the branch portion. For example, the valve 12 is configured as a solenoid valve. A pinch valve, a syringe, or the like may be used as the valve 12 in place of a solenoid valve.

The controller 11 is configured to control opening and closing of the valve 12. The valve 12 is configured to open the water suction tube 54 to atmosphere in an open state and to obstruct the branch portion in a closed state.

The primary strainer 55 and the secondary strainer 56 are filters configured to filter stones and mucous membranes suctioned from inside the perfusion object 90. Among the strainers, for example, the primary strainer 55 is used to collect stones. For example, the primary strainer 55 is mounted to a distal end side of the grasping portion 32a in the operating unit 32 of the endoscope 30. However, the primary strainer 55 and the secondary strainer 56 are not limited to the arrangement shown in FIG. 1 and only one may or may not be provided.

The water suction pressure gauge 16 is installed between, for example, the branch portion to the valve 12 and the water suction flow rate meter 18 between the water suction pump 14 and the water suction channel 35 on the water suction tube 54. The water suction pressure gauge 16 includes a pressure sensor or the like and is configured to detect pressure of a fluid inside the water suction tube 54. The processor 11A of the controller 11 is configured to acquire pressure detected by the water suction pressure gauge 16.

Of the third water suction tube 54c, a first end is connected to the secondary strainer 56 and another end is connected to the wastewater tank 52 via the water suction pump 14.

The water suction pump 14 is connected to the water suction tube 54. The water suction pump 14 is configured to suction the liquid inside the perfusion object 90 together with stones and to feed the liquid and the stones toward a side of the wastewater tank 52 via the water suction tube 54.

The wastewater tank 52 is configured to store the liquid from which stones and the like have been filtered by the primary strainer 55 and the secondary strainer 56.

The water feed pump 13 and the water suction pump 14 are respectively driven by the controller 11. When the water feed pump 13 and the water suction pump 14 are operated, water feeding to the inside of the perfusion object 90 and suction of the liquid inside the perfusion object 90 are simultaneously performed.

As a result, the liquid discharged from the water feed channel 34 creates a circulating flow (perfusion) inside the perfusion object 90 and fragmented stones are carried by the flow. Accordingly, the liquid inside the perfusion object 90 is suctioned together with the stones from the channel opening 35a of the water suction channel 35 and collection efficiency of stones improves.

The water feed flow rate meter 17 is provided on the water feed tube 53 and is configured to detect a water feed flow rate in the water feed tube 53. The processor 11A of the controller 11 is configured to acquire a water feed flow rate detected by the water feed flow rate meter 17.

The water suction flow rate meter 18 is provided on the water suction tube 54 and is configured to detect a water suction flow rate in the water suction tube 54. The processor 11A is configured to acquire a water suction flow rate detected by the water suction flow rate meter 18.

The controller 11 includes the processor 11A and a memory 11B.

For example, the processor 11A includes hardware such as an ASIC (application specific integrated circuit) including a CPU (central processing unit) or the like or an FPGA (field programmable gate array).

The memory 11B is a recording medium (or a storage medium, a recording apparatus, or a storage apparatus; the same applies hereafter) configured to record (or store; the same applies hereafter) a processing program to be executed by the processor 11A, various setting values, and the like.

While an example will be described in which functions of respective units are fulfilled by having the processor 11A read and execute the processing program recorded in the memory 11B, the functions of the respective units are not limited thereto. For example, at least a part of the functions of the respective units fulfilled by the controller 11 may be configured as a dedicated electronic circuit.

FIG. 4 is a block diagram showing a functional configuration of the controller 11 according to the first embodiment.

For example, as functional units, the controller 11 includes a pressure reference creating unit 11a, a water feed abnormality detecting unit 11b, a water suction abnormality detecting unit 11c, a clogging detecting unit 11d, an internal pressure sensor abnormality detecting unit 11e, an internal pressure estimating unit 11f, a pump control unit 11g, and a correlation data specifying unit 11h. Configurations of the respective functional units will be sequentially described.

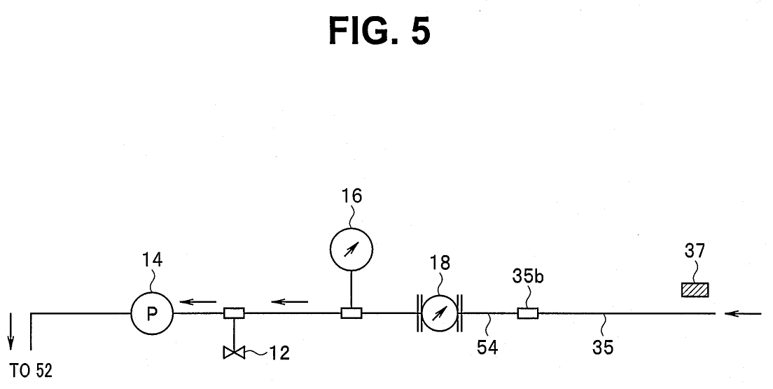

FIG. 5 is a diagram schematically showing a flow of a fluid of the water suction system in the endoscope fluid control system 1 in a state where the valve 12 is closed in the first embodiment.

An internal pressure sensor 37 configured to measure pressure (referred to as internal pressure when appropriate) inside the perfusion object 90 is arranged in a vicinity of, for example, the channel opening 35a of the water suction channel 35 in the distal end portion 31a of the insertion portion 31 of the endoscope 30.

For example, a piezoresistive sensor or an optical fiber sensor utilizing Fabry-Perot interference can be used as the internal pressure sensor 37. These sensors are extremely small and relatively expensive. In addition, a pressure sensor in which a tube connected thereto extends from a distance may be used as the internal pressure sensor 37.

The processor 11A is configured to acquire pressure (internal pressure) measured by the internal pressure sensor 37.

Since the water suction channel 35 is provided inside the insertion portion 31 of the endoscope 30, an inner diameter of the water suction channel 35 is assumed to be smaller than an inner diameter of the water suction tube 54. The water suction flow rate meter 18, the water suction pressure gauge 16, the valve 12, and the water suction pump 14 are arranged along a flow path on the water suction tube 54.

When the valve 12 is in the closed state, inside of a conduit of the water suction system is placed under negative pressure due to a suction force of the water suction pump 14. Therefore, a fluid inside the perfusion object 90 is suctioned and drained.

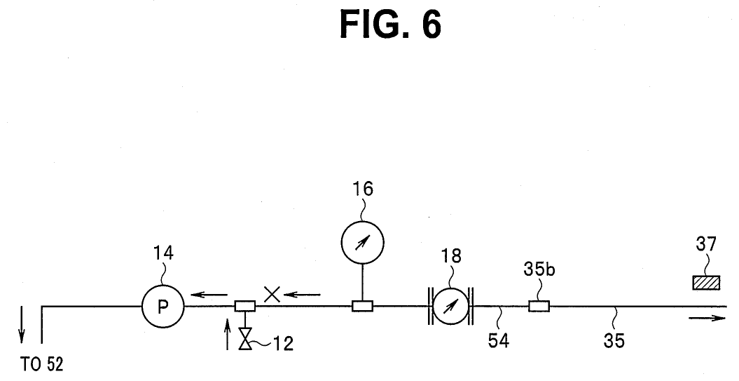

FIG. 6 is a diagram schematically showing a flow of a fluid of the water suction system in the endoscope fluid control system 1 in a state where the valve 12 is open in the first embodiment.

On the other hand, when the valve 12 opens during water suction, air flows from the valve 12 into the conduit inside the water suction system under negative pressure and flows toward the side of the water suction pump 14. Therefore, the flow of the fluid stops on an upstream side (a side of the water suction channel 35) of the branch portion of the valve 12. However, since a force of inertia acts on the fluid, an inflow to the branch portion of the valve 12 continues and a localized high-pressure portion is formed. Due to the high-pressure portion, as described above, a water hammer effect occurs in which the fluid inside the water suction channel 35 flows in reverse (jets in reverse).

When a water hammer effect occurs, a stone snagged in a conduit of the water suction system is detached from the conduit of the water suction system due to pressure on the fluid caused by the reverse jet. In consideration thereof, the controller 11 appropriately (for example, regularly) performs control of restoring the closed state after instantaneously creating the open state of the valve 12 to resolve snagging of a stone in the conduit of the water suction system.

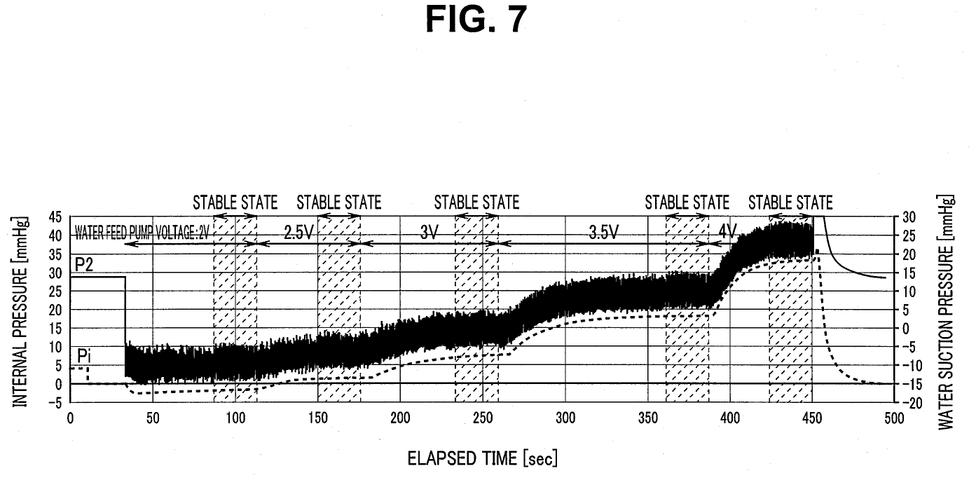

FIG. 7 is a graph showing a change over time of a pressure value Pi measured by the internal pressure sensor 37 and a pressure value P2 measured by the water suction pressure gauge 16 when performing water feeding and water suction to the perfusion object 90 in the first embodiment. In FIG. 7, a horizontal axis represents elapsed time, a vertical axis on a left side represents a scale of the pressure value Pi (depicted by a dotted line) measured by the internal pressure sensor 37, and a vertical axis on a right side represents a scale of the pressure value P2 (depicted by a solid line) measured by the water suction pressure gauge 16.

The pressure values Pi and P2 shown in FIG. 7 represent an example of values when the water suction pump 14 is operated under a constant operation condition (constant voltage) and the water feed pump 13 is operated at an operation condition that changes in stages (voltage is increased in stages from 2 V to 2.5 V to 3 V to 3.5 V to 4 V and the like). Furthermore, FIG. 7 also illustrates a region where pressure stabilizes under each operation condition.

As shown on the left-side vertical axis of FIG. 7, the pressure value Pi is basically set to positive pressure in order to inflate the inside of the perfusion object 90 and secure a field of view.

In addition, as shown on the right-side vertical axis of FIG. 7, the water suction system is basically set to negative pressure in order to suction (perform water suction of) a fluid.

As is apparent from the graph shown in FIG. 7, the pressure value Pi of internal pressure and the pressure value P2 of water suction pressure exhibit behaviors that are more or less parallel to each other.

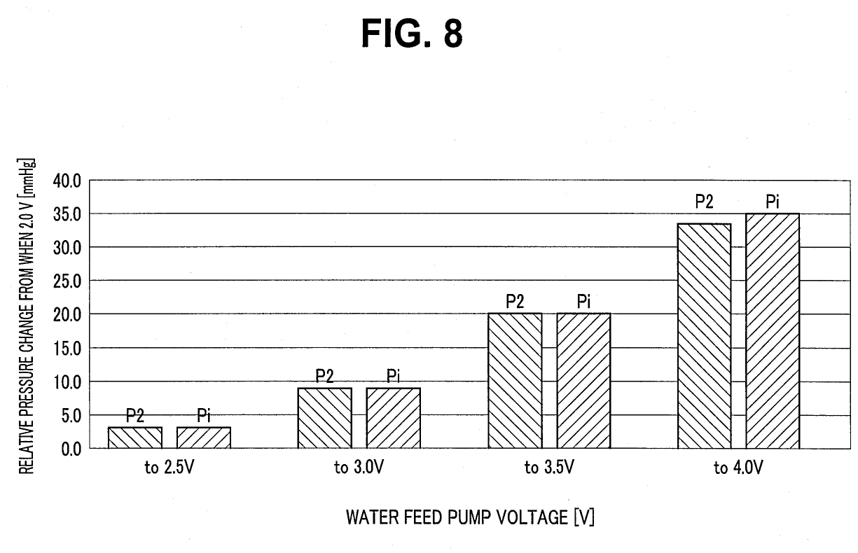

FIG. 8 is a bar chart showing a relative pressure change amount of the pressure value Pi and the pressure value P2 in a range in a stable state when an operation condition involving setting a drive voltage of the water feed pump 13 to 2 V is changed to an operation condition involving other drive voltages (2.5 V, 3.0 V, 3.5 V, and 4.0 V) in the graph shown in FIG. 7 according to the first embodiment.

As is apparent from FIG. 8, the pressure value Pi and the pressure value P2 exhibit excellent correlation. In other words, between the internal pressure sensor 37 and the water suction pressure gauge 16, while absolute values of measured pressure differ from each other, relative values of pressure can be compared under a suitable condition such as a stable state.

In consideration thereof, in the present embodiment, whether or not the internal pressure sensor 37 is normal is determined using the pressure value P2 measured by the water suction pressure gauge 16 based on a correlation between the pressure value Pi and the pressure value P2 to ensure that the internal pressure sensor 37 is operating normally.

Conversely, whether or not the water suction pressure gauge 16 is normal may be determined using the pressure value Pi measured by the internal pressure sensor 37 based on the correlation to ensure that the water suction pressure gauge 16 is operating normally.

### [Definition of Normal State]

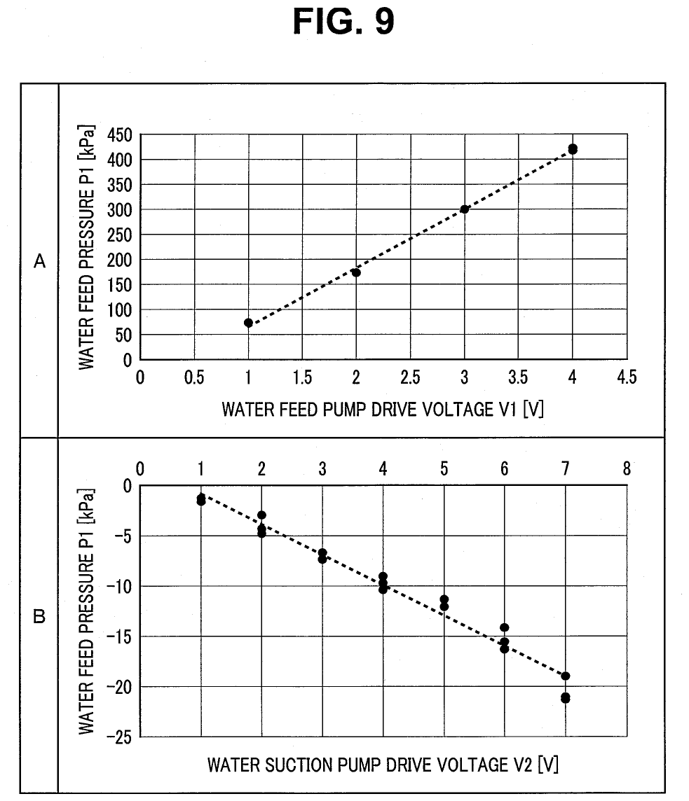

FIG. 9 is a chart in which graphs indicating a relationship between a drive voltage of a pump and a pressure value measured by a pressure gauge in the water feed system and the water suction system are arranged in the first embodiment.

A field A in FIG. 9 shows a graph presenting a relationship between a drive voltage V1 of the water feed pump 13 and a pressure value P1 measured by the water feed pressure gauge 15. When the drive voltage V1 increases, since a water feed flow rate increases, the pressure value P1 also increases.

A field B in FIG. 9 shows a graph presenting a relationship between a drive voltage V2 of the water suction pump 14 and the pressure value P2 measured by the water suction pressure gauge 16. When the drive voltage V2 increases, since a water suction flow rate increases, the pressure value P2 decreases.

In each of the fields A and B in FIG. 9, a dot depicts a pressure value and a straight dotted line depicts an optimization straight line that is obtained from a pressure value.

As shown in FIG. 9, in both the water feed system and the water suction system, for example, there is a linear correlation between the drive voltage of a pump (which corresponds to an output of the pump) and the measured pressure value in a stable state. Therefore, the water feed system can be determined to be normal when the pressure value P1 at the drive voltage V1 is on the optimization straight line. In addition, the water suction system can be determined to be normal when the pressure value P2 at the drive voltage V2 is on the optimization straight line. In this manner, in the example shown in FIG. 9, the optimization straight line indicates a normal range of each system (a region indicating that each system is in a normal state).

However, there may be cases where the water feed system and the water suction system can be determined to be normal even when a pressure value is not located on an optimization straight line. This is because variability occurs in the correlation shown in FIG. 9 due to individual variability of the pressure gauges 15 and 16, variations in the water feed system and the water suction system such as a change in conduit resistance due to an attitude of a conduit in the water feed system and the water suction system, and the like.

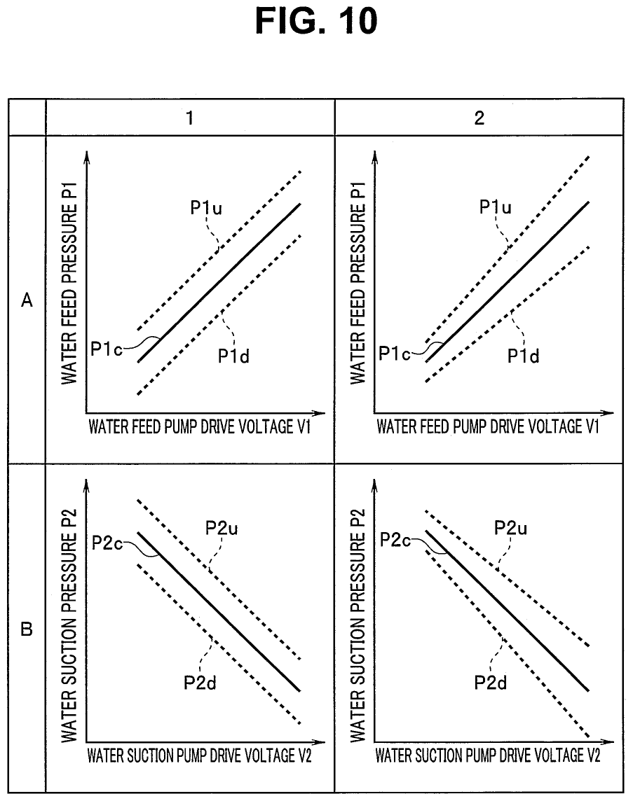

In consideration thereof, an example of defining a normal range with a certain amount of leeway is shown in FIG. 10.

FIG. 10 is a chart in which graphs indicating a normal range in a relationship between a drive voltage of a pump and a pressure value measured by a pressure gauge in the water feed system and the water suction system are arranged in the first embodiment. A field A in FIG. 10 shows two examples of a graph presenting a normal range of a relationship between a drive voltage V1 and a pressure value P1. A field B in FIG. 10 shows two examples of a graph presenting a normal range of a relationship between a drive voltage V2 and a pressure value P2.

Reference character P1c depicted by a straight line in field A in FIG. 10 denotes a graph of a pressure reference line (for example, corresponding to the optimization straight line in field A in FIG. 9) indicating a standard normal relationship of the pressure value P1 with respect to the drive voltage V1. Reference character P1u denotes a graph of a pressure upper limit line presenting an upper limit value of the normal range of the pressure value P1 with respect to the drive voltage V1. Reference character P1d denotes a graph of a pressure lower limit line presenting a lower limit value of the normal range of the pressure value P1 with respect to the drive voltage V1.

In the example shown in field A1 in FIG. 10, (P1u−P1c) and (P1c−P1d) assume constant values regardless of the drive voltage V1. Note that (P1u−P1c) and (P1c−P1d) need not be a same constant value and may respectively be different constant values. In addition, the fluid control apparatus for endoscope 10 may be configured to have a mode which enables a user or a worker to perform a maintenance checkup of the system to change (P1u−P1c) and (P1c−P1d).

In the example shown in field A2 in FIG. 10, (P1u−P1c) and (P1c−P1d) assume values that increase as the drive voltage V1 increases. Note that (P1u−P1c) and (P1c−P1d) need not be a same value at the same drive voltage V1 and may respectively be different values.

Reference character P2c depicted by a straight line in field B in FIG. 10 denotes a graph of a pressure reference line (for example, corresponding to the optimization straight line in field B in FIG. 9) indicating a standard normal relationship of the pressure value P2 with respect to the drive voltage V2. Reference character P2u denotes a graph of a pressure upper limit line presenting an upper limit value of the normal range of the pressure value P2 with respect to the drive voltage V2. Reference character P2d denotes a graph of a pressure lower limit line presenting a lower limit value of the normal range of the pressure value P2 with respect to the drive voltage V2.

In the example shown in field B1 in FIG. 10, (P2u−P2c) and (P2c−P2d) assume constant values regardless of the drive voltage V2. Note that (P2u−P2c) and (P2c−P2d) need not be a same constant value and may respectively be different constant values. In addition, the fluid control apparatus for endoscope 10 may be configured to have a mode which enables a user or a worker to perform a maintenance checkup of the system to change (P2u−P2c) and (P2c−P2d).

In the example shown in field B2 in FIG. 10, (P2u−P2c) and (P2c−P2d) assume values that increase as the drive voltage V2 increases. Note that (P2u−P2c) and (P2c−P2d) need not be a same value at the same drive voltage V2 and may respectively be different values.

As shown in field A2 and field B2 in FIG. 10, by changing the upper limit and the lower limit of the normal range in accordance with the drive voltages V1 and V2, a degree of pressure change during an abnormality which varies in accordance with the drive voltages V1 and V2 can be accommodated and whether or not a value is within the normal range can be determined more suitably.

While a pump drive voltage is plotted on the horizontal axis as coordinates indicating an output value of a pump in each graph shown in FIG. 9 and FIG. 10, the output value of a pump is not limited thereto. A flow rate per unit time of the pump or a rotating speed of the pump may be used as a value indicating an output value of the pump instead of the pump drive voltage. This is because the pump drive voltage, the flow rate per unit time of the pump, and the rotating speed of the pump are in an approximately proportional relationship and are mutually convertible.

Information on the normal range shown in FIG. 9 or FIG. 10 may be recorded in advance in, for example, the memory 11B.

An endoscope 30 of a different type (a different model, a different production lot, or the like) may be connected to a single fluid control apparatus for endoscope 10 depending on an examination. In such a case, variations of conduits of the water feed system and/or the water suction system are to be present in accordance with a type of the endoscope 30. Therefore, the normal range (correlation data) shown in FIG. 9 or FIG. 10 may be recorded in advance in the memory 11B for each variation (each type of the endoscope 30). Correlation data refers to data representing a correlation between a pressure value indicated by the water suction pressure gauge 16 (or the water feed pressure gauge 15) and an output value of the water suction pump 14 (or the water feed pump 13).

In this case, the controller 11 is to recognize a type of the endoscope 30 to be connected to the fluid control apparatus for endoscope 10 and select and read one normal range (correlation data) corresponding to the recognized type from the memory 11B. The type of the endoscope 30 may be recognized by the controller 11 based on type information transmitted from an input apparatus such as an operating switch in accordance with an operation by the user.

Alternatively, a non-volatile memory such as an EEPROM (electrically erasable and programmable read only memory) may be provided in the endoscope 30, type information of the endoscope 30 may be recorded in the non-volatile memory, and the processor 11A may read (acquire) the type information from the non-volatile memory of the endoscope 30. The processor 11A determines the type of the endoscope 30 from the acquired type information.

Furthermore, the endoscope 30 may be provided with an RFID (radio frequency identification) tag on which identification data including type information is written, the fluid control apparatus for endoscope 10 may be provided with an RFID reader, and the processor 11A may read (acquire) the type information using the RFID reader. The processor 11A determines the type of the endoscope 30 from the acquired type information.

The acquisition of type information is not limited to the examples described above and techniques that enable the processor 11A to directly or indirectly read (acquire) type information from the endoscope 30 connected to the fluid control apparatus for endoscope 10 may be used when appropriate.

The correlation data specifying unit 11h of the controller 11 is configured to specify correlation data corresponding to the type of the endoscope 30 (the endoscope 30 the water suction channel 35 of which is connected to the water suction tube 54) from among the correlation data set for each type of the endoscope 30 recorded in the memory 11B.

The controller 11 is configured to monitor an operation of the water suction pump 14 based on the correlation data specified by the correlation data specifying unit 11h (or correlation data calculated (constructed) as shown in equation (1) to be described later by the processor 11A).

In addition, a part of or all of the information on the normal range shown in FIG. 9 and FIG. 10 may be acquired when performing an endoscopy.

The pressure values P1 and P2 in accordance with the pump drive voltages V1 and V2 (or flow rates or rotating speeds as described above) are affected by conduits. In other words, to be exact, the pressure values P1 and P2 are affected by a difference between types of the tubes 53 and 54 connected to the pumps 13 and 14, a difference between types of endoscopes 30, and the like. In particular, when detecting a relatively small pressure change such as when detecting clogging of a conduit, the effect of such a difference between types can be non-negligible.

In consideration thereof, by connecting the tubes 53 and 54 and the endoscope 30 to the fluid control apparatus for endoscope 10 and defining a normal state in a state where installation of the endoscope fluid control system 1 has been completed, effects of differences between types and combinations can be eliminated.

As shown in FIG. 9 and FIG. 10, basically, the pressure reference lines P1c and P2c representing a standard normal relationship can be linearly approximated and represented by primary straight lines. Therefore, the pressure reference lines P1c and P2c can be defined by acquiring at least two coordinate points determined by a combination of a drive voltage of a pump and a pressure value measured by a pressure gauge.

However, definitions are not limited thereto and when it is known in advance that an approximated curve such as a multidimensional linear approximation, an exponential approximation, or a logarithmic approximation is desirably used as a curved line formed by a pressure value measured by a pressure gauge with respect to a drive voltage of a pump, each curve can be defined by acquiring data of a plurality of points necessary for determining each approximated curve.

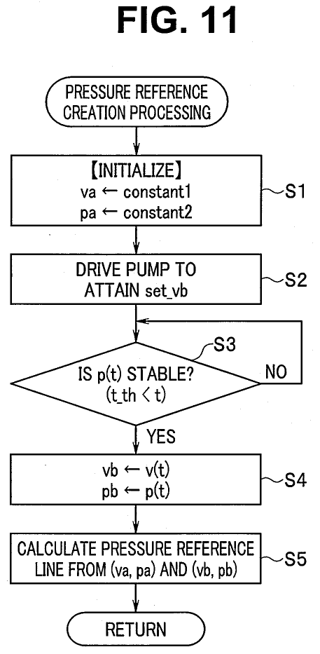

In a realistic medical setting, it is difficult to define a normal state over a long period of time and the normal state is desirably defined as easily as possible. In consideration thereof, as will be described below with reference to FIG. 11, data of points less likely to be affected by differences in types may be recorded in the memory 11B in advance and data of only one point may be acquired in a medical setting. Accordingly, a normal state that takes differences in types into consideration can be defined in a short period of time.

FIG. 11 is a flowchart showing pressure reference creation processing by the endoscope fluid control system 1 according to the first embodiment. The processing shown in FIG. 11 is executed by the pressure reference creating unit 11a of the controller 11.

When the processing is started, the processor 11A performs initialization of assigning a value (constant1, constant2) recorded in the memory 11B in advance as a value of a first point (va, pa) (step S1).

In the case of the water suction system, reference character va represents a first value of the drive voltage V1 of the water suction pump 14 and reference character pa represents a pressure value measured by the water suction pressure gauge 16 when drive voltage V1=va. In addition, in the case of the water feed system, reference character va represents a first value of the drive voltage V2 of the water feed pump 13 and reference character pa represents a pressure value measured by the water feed pressure gauge 15 when drive voltage V2=va. In other words, the memory 11B is configured to record, at least for each type of the endoscope 30, first minimum correlation data that is constructed based on a first pressure value pa indicated by the pressure gauge 15 or 16 and an output value (drive voltage va) of the pump 13 or 14 corresponding to the first pressure value pa. Hereinafter, the water suction system will be described as an example for the sake of simplicity.

Generally, the larger the drive voltage or, in other words, the larger the flow rate, the larger the variation among measured pressure values. Therefore, the value (constant1, constant2) recorded in the memory 11B in advance is a value (first minimum correlation data) corresponding to a state of a small flow rate (for example, a flow rate of 10 (mL/min)).

The memory 11B is configured to record a plurality of types of the first minimum correlation data in which the first pressure value indicated by the water suction pressure gauge 16 or the output value of the water suction pump 14 differ from each other. The processor 11A of the controller 11 is configured to select and read a type of minimum correlation data corresponding to the type of the endoscope 30 from among the plurality of types of the first minimum correlation data recorded in the memory 11B.

When there is time to spare at a medical site, the first point (va, pa) may be acquired by measurement.

Next, the processor 11A drives the pump so that the drive voltage reaches a given target value set_vb (step S2). In this case, the target value set_vb is a drive voltage corresponding to a maximum value (for example, 40 (mL/min)) of a flow rate that is assumed in a medical setting. The target value set_vb may be a value determined in advance or a value set by the user.

The processor 11A monitors a pressure value p(t) measured by the pressure gauge during an elapsed time period t after the pump is driven at the target value set_vb and determines whether or not p(t) is stable (step S3).

Whether or not p(t) is stable may be determined based on whether or not the elapsed time period t is longer than a predetermined threshold time period t_th necessary for stabilization. The predetermined threshold time period t_th is a time period determined in advance as a time period necessary for stabilization.

In addition, the processor 11A may be configured to determine that p(t) has stabilized when a deviation or a variation between the present pressure value p(t) and a pressure value at a predetermined time before the present becomes smaller than a predetermined value. Alternatively, the processor 11A may be configured to determine stability of p(t) based on a transfer function of the system.

Furthermore, a change over time of p(t) may be displayed on, for example, the display apparatus 25 as a graph or a change in a numerical value and the user viewing the display apparatus 25 may determine whether or not p(t) has stabilized.

When p(t) is not stabilized, the processor 11A stands by until p(t) stabilizes.

On the other hand, when p(t) is stabilized in step S3, the processor 11A assigns a value (v(t), p(t)) when stable as a value of a second point (vb, pb) (step S4). The second point (vb, pb) to which the value (v(t), p(t)) when stable is assigned represents second minimum correlation data. In other words, the processor 11A is configured to construct the second minimum correlation data based on a second pressure value pb measured by the water suction pressure gauge 16 and the output value (drive voltage vb) of the water suction pump 14 corresponding to the second pressure value pb when the water suction channel 35 is connected to the water suction tube 54 and the water suction pump 14 is operated.

In addition, the processor 11A calculates a straight line that passes through the first point (va, pa) and the second point (vb, pb) (a straight line that provides pressure P_{std} with respect to a drive voltage V_{std}) as shown in equation (1) and adopts the straight line as a pressure reference line (step S5). In this case, the processor 11A constructs correlation data (pressure reference line) based on a combination of the first minimum correlation data (the first point (va, pa)) and the second minimum correlation data (the second point (vb, pb)).

$${P}_{std} = \frac{pb - pa}{vb - va} \times {V}_{std} + \frac{vb \times pa - va \times pb}{vb - va} \qquad ( 1 )$$

When correlation data set for each type of the endoscope 30 is recorded in the memory 11B, the processor 11A may be configured to specify correlation data which approximates the combined data of the first minimum correlation data and the second minimum correlation data from among the correlation data set for each type of the endoscope 30 which is recorded in the memory 11B.

Accordingly, since the pressure reference line with respect to a drive voltage (or a flow rate or a rotating speed as described above) has been defined, the processor 11A returns processing to main processing (not illustrated) or the like.

### [Water Feeding/Water Suction Abnormality Detection]

When the relationship between a drive voltage and a pressure value deviates from the normal range described above, an occurrence of an abnormal state such as an occurrence of buckling of a conduit or an occurrence of a leakage midway along a conduit due to a poor connection can be detected.

In order to suitably determine that an abnormal state has occurred, a transient state such as upon start-up or when a setting has been changed may be taken into consideration. Upon start-up or when a setting has been changed, a time period in accordance with each circumstance is required until the relationship between the drive voltage and the pressure value reaches a steady state. A water feed abnormality and a water suction abnormality can be suitably detected by taking such a transient state into consideration.

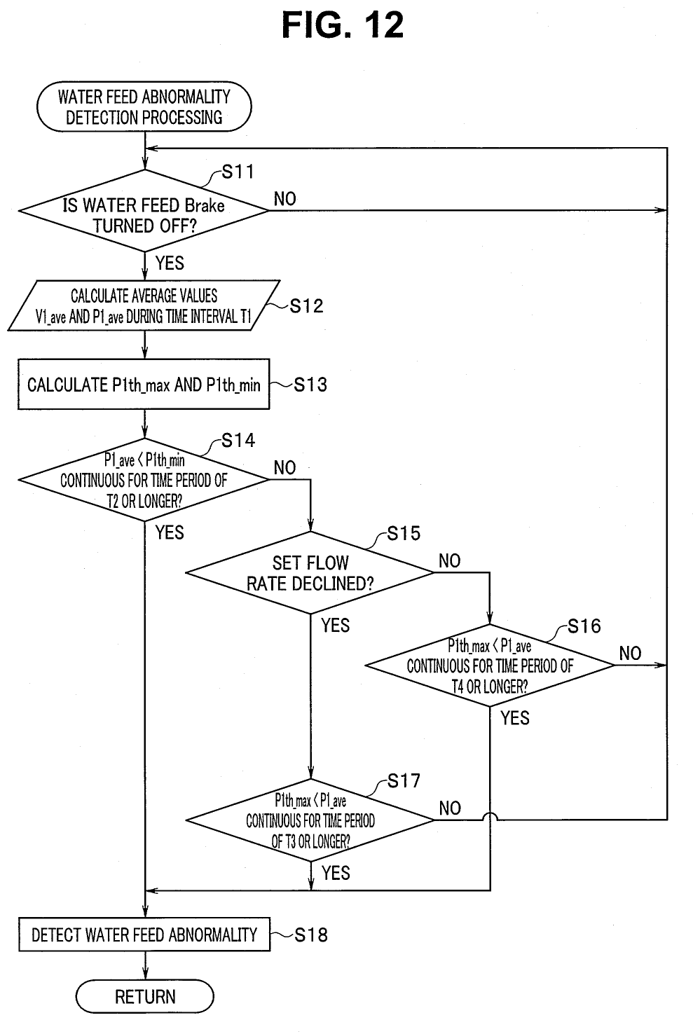

FIG. 12 is a flowchart showing water feed abnormality detection processing according to the first embodiment. The processing shown in FIG. 12 is executed by the water feed abnormality detecting unit 11b of the controller 11.

When the water feed abnormality detection processing is started, the processor 11A determines whether or not water feed Brake is turned off (step S11). In this case, water feed Brake is a signal for stopping the water feed pump 13. When water feed Brake is turned on, since the water feed pump 13 has stopped, the processor 11A returns to processing at a start time point of the water feed abnormality detection processing and stands by until water feed Brake is turned off.

On the other hand, when water feed Brake is turned off, the processor 11A calculates a moving average value V1_ave of a drive voltage V1 of the water feed pump 13 (indicated voltage to the water feed pump 13) and a moving average value P1_ave of a pressure value P1 measured by the water feed pressure gauge 15 during a predetermined time interval T1 (step S12).

The time interval T1 is a time period for performing averaging in consideration of a variation in data (raw data) that is sequentially acquired as time elapses. While a suitable value of the time interval T1 differs in accordance with a frequency at which data is to be sampled, for example, a value of around 1 second is desirable.

A case is now assumed where the normal range has a constant width in upward and downward directions centered on the pressure reference line P1c as shown in field A1 in FIG. 10. In this case, a pressure upper limit line P1u is obtained by adding a constant value (P1u−P1c) to the pressure reference line P1c. In addition, a pressure lower limit line P1d is obtained by subtracting a constant value (P1c−P1d) from the pressure reference line P1c.

In consideration thereof, the processor 11A adds the constant value (P1u−P1c) to a pressure value obtained by substituting the moving average value V1_ave into the pressure reference line P1c created by the processing shown in FIG. 11 to calculate an upper limit value P1th_max of the normal range. Furthermore, the processor 11A subtracts the constant value (P1c−P1d) from a pressure value obtained by substituting the moving average value V1_ave into the pressure reference line P1c created by the processing shown in FIG. 11 to calculate a lower limit value P1th_min of the normal range (step S13).

In addition, the processor 11A determines whether or not a state where P1_ave<P1th_min is satisfied is continuing for a predetermined determination time period of T2 or longer (step S14).

The determination time period T2 is an upper limit threshold of a time period required by the moving average value V1_ave that is lower than the lower limit value P1th_min to reach the lower limit value P1th_min of the normal range by a transient response. The determination time period T2 is a time period necessary for determining whether a transient response has been made or an abnormality has occurred. While a time period suitable as the determination time period T2 depends on a conduit resistance, a responsiveness of a fluid system until the pressure value P1 rises, or the like, for example, the time period is 40 seconds.

When the state where P1_ave<P1th_min is satisfied is not continuing for the determination time period of T2 or longer, the processor 11A determines whether or not a present flow rate set to the water feed pump 13 or, in other words, the present drive voltage V1 of the water feed pump 13 has decreased as compared to before (step S15).

When the drive voltage V1 has not decreased, the processor 11A determines whether or not a state where P1th_max<P1_ave is satisfied is continuing for a predetermined determination time period of T4 or longer (step S16). The determination time period T4 is a threshold of a time period during which the moving average value P1_ave is exceeding the upper limit value P1th_max of the normal range. The determination time period T4 is set to a short time period since, desirably, a time period during which the moving average value P1_ave is exceeding the upper limit value P1th_max of the normal range is not increased. A time period suitable as the determination time period T4 is, for example, 1 second.

When the state where P1th_max<P1_ave is satisfied is not continuing for the determination time period of T4 or longer, the processor 11A returns to processing at a start time point of the water feed abnormality detection processing.

When the drive voltage V1 has decreased in step S15, the processor 11A determines whether or not a state where P1th_max<P1_ave is satisfied is continuing for a predetermined determination time period of T3 or longer (step S17).

The determination time period T3 is an upper limit threshold of a time period required by the moving average value V1_ave that is larger than the upper limit value P1th_max to reach the upper limit value P1th_max of the normal range by a transient response when the drive voltage V1 (and therefore, the set flow rate) decreases. The determination time period T3 is a time period necessary for determining whether a transient response has been made or an abnormality has occurred. While a time period suitable as the determination time period T3 depends on a conduit resistance, a responsiveness of a fluid system until the pressure value P1 falls, or the like, for example, the time period is 20 seconds.

When the state where P1th_max<P1_ave is satisfied is not continuing for the determination time period of T3 or longer, the processor 11A returns to processing at the start time point of the water feed abnormality detection processing.

When the state where P1_ave<P1th_min is satisfied is continuing for the determination time period of T2 or longer in step S14, when the state where P1th_max<P1_ave is satisfied is continuing for the determination time period of T4 or longer in step S16, or when the state where P1th_max<P1_ave is satisfied is continuing for the determination time period of T3 or longer in step S17, the processor 11A detects that a water feed abnormality has occurred (step S18).

When the processor 11A detects that a water feed abnormality has occurred, the processor 11A returns processing to main processing (not illustrated) or the like and, for example, executes separate processing or the like corresponding to the water feed abnormality. An example of separate processing corresponding to the water feed abnormality is processing of stopping a pump system and notifying and requesting the user using at least one of a text, color, sound, or the like to check whether or not an abnormality is absent in the water feed conduit because a water feed abnormality has occurred.

The fluid control apparatus for endoscope 10 may be configured to have a mode which enables a user or a worker to perform a maintenance checkup of the system to change the time interval T1 and the determination time periods T2 to T4 described above.

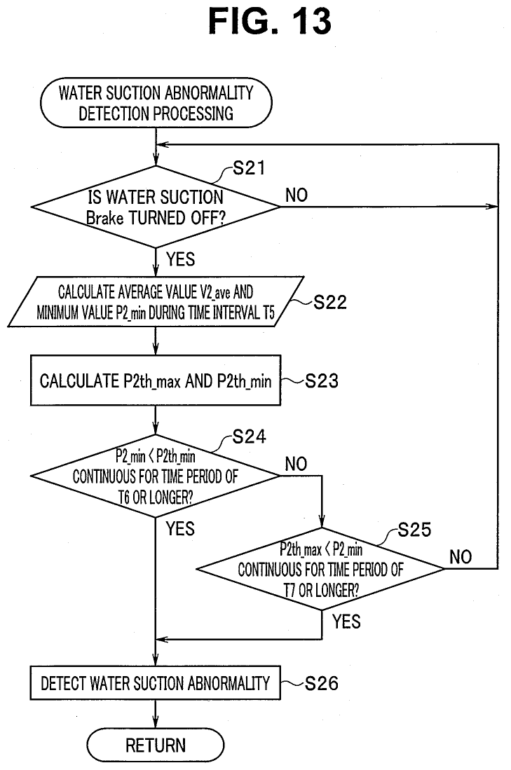

FIG. 13 is a flowchart showing water suction abnormality detection processing according to the first embodiment. The processing shown in FIG. 13 is executed by the water suction abnormality detecting unit 11c of the controller 11.

When the water suction abnormality detection processing is started, the processor 11A determines whether or not water suction Brake is turned off (step S21). In this case, water suction Brake is a signal for stopping the water suction pump 14. When water suction Brake is turned on, since the water suction pump 14 has stopped, the processor 11A returns to processing at a start time point of the water suction abnormality detection processing and stands by until water suction Brake is turned off.

On the other hand, when water suction Brake is turned off, the processor 11A calculates a moving average value V2_ave of a drive voltage V2 of the water suction pump 14 during a predetermined time interval T5. In addition, the processor 11A acquires a minimum value P2_min of a pressure value P2 measured by the water suction pressure gauge 16 during the predetermined time interval T5 (step S22).

The time interval T5 is a time period for performing averaging in consideration of a variation in data (raw data) that is sequentially acquired as time elapses. The time interval T5 may be the same as the time interval T1 shown in FIG. 12. However, when removing clogging by generating a water hammer effect by periodically creating the opened state of the valve 12 shown in FIG. 6 in the closed state of the valve 12 shown in FIG. 5, a cycle time period of opening and closing the valve 12 is desirably set as the time interval T5. A time period suitable as the time interval T5 is, for example, 3 seconds. When a syringe is connected instead of the valve 12 and a flush operation by the syringe is to be periodically performed, the time interval T5 is adjusted to a cycle of the flush operation.

A case is now assumed where the normal range has a constant width in upward and downward directions centered on the pressure reference line P2c as shown in field B1 in FIG. 10. In this case, a pressure upper limit line P2u is obtained by adding a constant value (P2u−P2c) to the pressure reference line P2c. In addition, a pressure lower limit line P2d is obtained by subtracting a constant value (P2c−P2d) from the pressure reference line P2c.

In consideration thereof, the processor 11A adds the constant value (P2u−P2c) to a pressure value obtained by substituting the moving average value V2_ave into the pressure reference line P2c created by the processing shown in FIG. 11 to calculate an upper limit value P2th_max of the normal range. Furthermore, the processor 11A subtracts the constant value (P2c−P2d) from a pressure value obtained by substituting the moving average value V2_ave into the pressure reference line P2c created by the processing shown in FIG. 11 to calculate a lower limit value P2th_min of the normal range (step S23).

In addition, the processor 11A determines whether or not a state where P2_min<P2th_min is satisfied is continuing for a predetermined determination time period of T6 or longer (step S24). The determination time period T6 is a threshold of a time period in which a state of being under the lower limit value P2th_min of the normal range continues. Since a long determination time period T6 may possibly cause excessive water suction, a short time period is desirably set. A time period suitable as the determination time period T6 is, for example, 3 seconds.

When the state where P2_min<P2th_min is satisfied is not continuing for the determination time period of T6 or longer, the processor 11A determines whether or not a state where P2th_max<P2_min is satisfied is continuing for a predetermined determination time period of T7 or longer (step S25). The determination time period T7 is a threshold of a time period during which the minimum value P2_min is exceeding the upper limit value P2th_max of the normal range. The determination time period T7 is a time period necessary for distinguishing whether the state in which the minimum value P2_min is exceeding the upper limit value P2th_max of the normal range is a transient state or an abnormal state. Since the distinguishing requires a certain amount of time, a time period suitable as the determination time period T7 is, for example, 40 seconds.

When the state where P2th_max<P2_min is satisfied is not continuing for the determination time period of T7 or longer, the processor 11A returns to processing at the start time point of the water suction abnormality detection processing.

When the state where P2_min<P2th_min is satisfied is continuing for the determination time period of T6 or longer in step S24 or when the state where P2th_max<P2_min is satisfied is continuing for the determination time period of T7 or longer in step S25, the processor 11A detects that a water suction abnormality has occurred (step S26).

When the processor 11A detects that a water suction abnormality has occurred, the processor 11A returns processing to main processing (not illustrated) or the like and, for example, executes separate processing or the like corresponding to the water suction abnormality. An example of separate processing corresponding to the water suction abnormality is processing of stopping a pump system and notifying and requesting the user using at least one of a text, color, sound, or the like to check whether or not an abnormality is absent in the water suction conduit because a water suction abnormality has occurred.

A determination based on the minimum value P2_min is performed above. This is because, given that a pressure value returns to near atmospheric pressure at a time interval (interval) of opening and closing the valve 12 in a configuration where clogging of the water suction conduit is prevented and removed by opening and closing the valve 12, a determination is desirably made using the pressure minimum value P2_min in the interval.

In contrast, in the case of a configuration where the valve 12 prevents and removes clogging of the water suction conduit by periodically generating a pressure fluctuation such as a syringe flush, a hand pump, or the like, a determination may be made based on the pressure minimum value P2_min or a determination may be made based on a pressure average value P2_ave.

The fluid control apparatus for endoscope 10 may be configured to have a mode which enables a user or a worker to perform a maintenance checkup of the system to change the time interval T5 and the determination time periods T6 and T7 described above.

### [Clogging Detection]

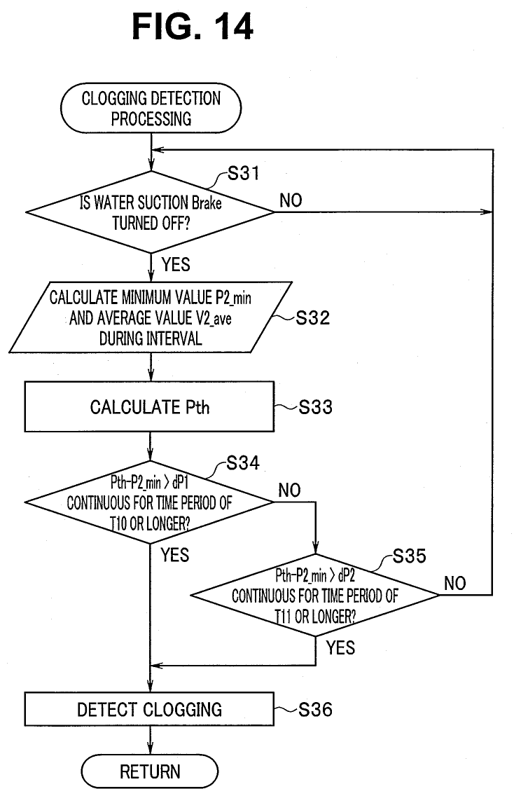

FIG. 14 is a flowchart showing clogging detection processing according to the first embodiment. The processing shown in FIG. 14 is executed by the clogging detecting unit 11d of the controller 11.

Clogging of the water suction tube 54 due to a stone can be detected using the normal ranges shown in FIG. 9 and FIG. 10. When a pressure drop from the normal range occurs by a predetermined amount or more and continues for a predetermined time period or longer during water suction, the processor 11A is configured to determine that clogging is occurring. Clogging is roughly divided into a type of clogging that occurs at a distal end of the water suction system and a type of clogging that occurs inside the water suction conduit.

Since a pressure drop that occurs during clogging differs depending on a type of the clogging, a detection condition in at least two stages is desirably set as will be described below. However, the detection condition may be divided into further levels depending on a degree of clogging in each type. In this case, since a detection condition in a larger number of stages is required, a detection condition in three or more stages may be set.

A pressure drop ((Pth−P2_min) to be described later) upon an occurrence of clogging has a relatively small value and, in some cases, the pressure drop is possibly a change in the normal range. In consideration thereof, the normal range may be contracted by bringing the pressure upper limit line P2u and the pressure lower limit line P2d closer to the pressure reference line P2c in order to enable clogging to be detected more suitably. In addition, a pressure drop may be monitored with the pressure reference line P2c as a reference. In doing so, a pressure drop amount at which an occurrence of clogging is detected may be changed in accordance with the drive voltage V2 (or a flow rate or a rotating speed as described above) of the water suction pump 14.

Hereinafter, an example of monitoring a pressure drop with the pressure reference line P2c as a reference will be described.

When the clogging detection processing is started, the processor 11A determines whether or not water suction Brake is turned off (step S31). At this point, when water suction Brake is turned on, the processor 11A stands by until water suction Brake is turned off.

On the other hand, when water suction Brake is turned off, the processor 11A calculates a moving average value V2_ave of the drive voltage V2 of the water suction pump 14 during a predetermined time interval (for example, a similar time interval T5 as in step S22). In addition, the processor 11A acquires a minimum value P2_min of a pressure value P2 measured by the water suction pressure gauge 16 during the predetermined time interval (step S32).

The processor 11A calculates a pressure value Pth obtained by substituting the moving average value V2_ave into the pressure reference line P2c created by the processing shown in FIG. 11 (step S33).

In addition, the processor 11A determines whether or not a state where (Pth−P2_min)>dP1 is satisfied is continuing for a predetermined determination time period of T10 or longer (step S34).

In this case, reference character dP1 denotes a threshold (a threshold of pressure for abnormality determination) set in advance with respect to a pressure drop from the pressure value Pth in order to detect severe clogging. In addition, the determination time period T10 is a threshold of a time period for detecting severe clogging. When detecting severe clogging, the threshold dP1 may be relatively large and the determination time period T10 may be relatively short. For example, the threshold dP1 is 3 (kPa) and the determination time period T10 is 0.1 seconds.

When the state where (Pth−P2_min)>dP1 is satisfied is not continuing for the determination time period of T10 or longer, the processor 11A determines whether or not a state where (Pth−P2_min)>dP2 is satisfied is continuing for a predetermined determination time period of T11 or longer (step S35).

In this case, reference character dP2 denotes a threshold (a threshold of pressure for abnormality determination) set in advance with respect to a pressure drop from the pressure value Pth in order to detect minor clogging. In addition, the determination time period T11 is a threshold of a time period for detecting minor clogging. When detecting minor clogging, the threshold dP2 is smaller than the threshold dP1 and the determination time period T11 is longer than the determination time period T10. For example, the threshold dP2 is 1 (kPa) and the determination time period T11 is 60 seconds.

When the state where (Pth−P2_min)>dP2 is satisfied is not continuing for the determination time period of T11 or longer, the processor 11A returns to processing at the start time point of the clogging detection processing.

When the state where (Pth−P2_min)>dP1 is satisfied is continuing for the determination time period of T10 or longer in step S34 or when the state where (Pth−P2_min)>dP2 is satisfied is continuing for the determination time period of T11 or longer in step S35, the processor 11A determines that clogging has been detected (step S36).

When clogging is detected, the processor 11A returns processing to main processing (not illustrated) or the like and, for example, executes separate processing or the like corresponding to the clogging. An example of separate processing corresponding to the clogging is processing of notifying and requesting the user using at least one of a text, color, sound, or the like to perform an operation of removing the clogging because clogging has occurred. The notification and the like described above may be performed after stopping the pump system. Alternatively, stoppage of the pump system may be selectively performed in accordance with a degree of clogging.

The fluid control apparatus for endoscope 10 may be configured to have a mode which enables a user or an operator to perform a maintenance checkup of the system to change the determination time periods T10 and T11 and the thresholds dP1 and dP2 described above.

### [Internal Pressure Sensor Abnormality Detection]

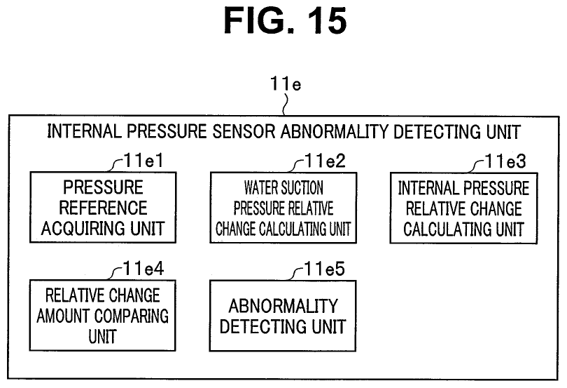

FIG. 15 is a block diagram showing a configuration example of the internal pressure sensor abnormality detecting unit 11e according to the first embodiment.

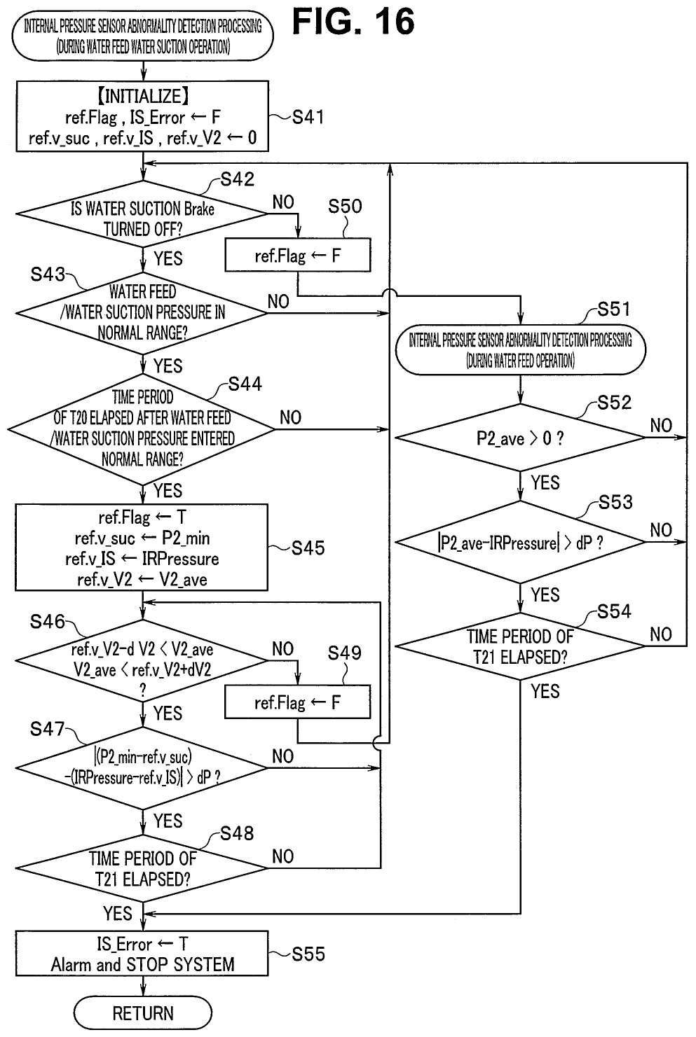

The internal pressure sensor abnormality detecting unit 11e includes a pressure reference acquiring unit 11e1, a water suction pressure relative change calculating unit 11e2, an internal pressure relative change calculating unit 11e3, a relative change amount comparing unit 11e4, and an abnormality notifying unit 11e5. A function of each unit will be described along the flowchart shown in FIG. 16.

FIG. 16 is a flowchart showing internal pressure sensor abnormality detection processing according to the first embodiment. The processing shown in FIG. 16 is executed by the internal pressure sensor abnormality detecting unit 11e of the controller 11. Hereinafter, logical values will be represented by F (false) and T (true).

The processor 11A first starts internal pressure sensor abnormality detection processing when the water feed pump 13 and the water suction pump 14 are operated.

The processor 11A then performs initialization of assigning F to a flag ref Flag, assigning F to a flag IS_Error, assigning 0 to a pressure value ref.v_suc, assigning 0 to a pressure value ref.v_IS, and assigning 0 to a voltage value ref.v_V2 (step S41).

In this case, the flag ref Flag is a flag indicating whether a pressure reference for calculating a relative change amount has been set (T) or not (F). The flag IS_Error is a flag indicating whether an abnormality of the internal pressure sensor 37 has been detected (T) or not (F). The pressure value ref._suc is a pressure value of the water suction pressure gauge 16 to be a reference when calculating a relative change amount. The pressure value ref.v_IS is a pressure value of the internal pressure sensor 37 to be a reference when calculating a relative change amount. The voltage value ref.v_V2 is a drive voltage of the water suction pump 14 when acquiring the pressure value ref.v_suc and the pressure value ref.v_IS.

The processor 11A determines whether or not water suction Brake is turned off (step S42).

[Creation of Pressure Reference] (Including Working of Pressure Reference Acquiring Unit 11e1]

At this point, when water suction Brake is turned off, the processor 11A determines whether a combination of the drive voltage V1 of the water feed pump 13 and the pressure value P1 measured by the water feed pressure gauge 15 is in the normal range such as shown in field A in FIG. 10 and, at the same time, a combination of the drive voltage V2 of the water suction pump 14 and the pressure value P2 measured by the water suction pressure gauge 16 is in the normal range such as shown in field B in FIG. 10 (step S43).

When the combinations are in the normal ranges in step S43, the processor 11A further determines whether a predetermined time period T20 has elapsed after entering the normal ranges (step S44).

A reference is necessary in order to obtain a relative change amount between the pressure value Pi measured by the internal pressure sensor 37 and the pressure value P2 measured by the water suction pressure gauge 16 as shown in FIG. 8. In the case of the internal pressure sensor 37, a pressure value measured at a given time point may be adopted as a reference. By comparison, in the case of the water suction pressure gauge 16, a pressure value measured when operations of the water feed pump 13 and the water suction pump 14 are stable must be used.

In consideration thereof, a determination that operation is stable is made when a state where the pressure value P2 measured by the water suction pressure gauge 16 is within the normal range as shown in FIG. 10 continues for the predetermined time period T20. The predetermined time period T20 is, for example, 30 seconds.

When the predetermined time period T20 elapses in step S44, the processor 11A assigns T to the flag ref Flag, assigns P2_min to the pressure value ref.v_suc, assigns IRPressure to the pressure value ref.v_IS, and assigns the moving average value V2_ave to the voltage value ref.v_V2 (step S45).

In this case, reference character P2_min denotes a minimum value of the pressure value P2 measured by the water suction pressure gauge 16 during a previous predetermined time period. Note that an average value P2_ave of the pressure value P2 measured by the water suction pressure gauge 16 during the previous predetermined time period may be used in place of P2_min. Reference character IRPressure denotes an average value of the pressure value Pi measured by the internal pressure sensor 37 during a predetermined time period. As described above, reference character V2_ave denotes a moving average value during a predetermined time period of the drive voltage V2 of the water suction pump 14.

As described above, according to the processing of steps S42 to S45, the pressure reference acquiring unit 11e1 creates a pressure reference. In contrast to the pressure reference creating unit 11a shown in FIG. 4 configured to create a pressure reference line or a normal range such as those shown in FIG. 9 and FIG. 10, the pressure reference acquiring unit 11e1 shown in FIG. 15 is configured to acquire a pressure value (in the example shown in FIG. 8, the pressure values P2 and Pi when a water feed pump voltage is 2.0 V) to be a reference in order to calculate a relative pressure change in FIG. 8.

### [Determination of Internal Pressure Sensor Abnormality]

In addition, the processor 11A determines whether (ref.v_V2−dV2)<V2_ave and V2_ave<(ref.v_V2+dV2) are satisfied (step S46).

In step S46, a determination is made as to whether or not a water suction condition at the time of creation of the reference has not changed based on whether or not a present moving average value V2_ave (or V2_min) of the drive voltage V2 of the water suction pump 14 is included in a predetermined range ref.v_V2±dV2 centered on a voltage value ref.v_V2 of the water suction pump 14 at the time of creation of the reference. Therefore, reference character dV2 denotes a predetermined value indicating a range for determining a presence or absence of a change in the water suction condition.

At this point, when the determination condition in step S46 is satisfied, the processor 11A determines whether or not |(P2_min−ref.v_suc)−(IRPressure−ref.v_IS)|>dP is satisfied (step S47). In this case, reference character dP denotes a threshold for determining whether or not a difference between the pressure value P2 measured by the water suction pressure gauge 16 and the pressure value Pi measured by the internal pressure sensor 37 is abnormal.

More specifically, the water suction pressure relative change calculating unit 11e2 calculates a first relative change amount (P2_min−ref.v_suc). The internal pressure relative change calculating unit 11e3 calculates a second relative change amount (IRPressure−ref.v_IS). In addition, the relative change amount comparing unit 11e4 performs the comparison determination in step S47.

In step S47, the first relative change amount (P2_min−ref.v_suc) of the minimum value P2_min of the pressure value P2 measured by the water suction pressure gauge 16 from the first reference value ref.v_suc and the second relative change amount (IRPressure−ref.v_IS) of the average value IRPressure of the pressure value Pi measured by the internal pressure sensor 37 from the second reference value ref.v_IS are compared with each other.

When a difference between the first relative change amount and the second relative change amount is equal to or smaller than the threshold dP (in other words, when the difference is small), both the water suction pressure gauge 16 and the internal pressure sensor 37 are normal. On the other hand, when the difference is larger than the threshold dP, at least one of the water suction pressure gauge 16 or the internal pressure sensor 37 (for example, the internal pressure sensor 37) is abnormal and has possibly failed.

Note that the threshold dP is desirably determined based on factors such as how accurately a relative change can be captured, an acceptable error necessary for ensuring safety, and the like. The threshold dP is, for example, 30 (mmHg).

When the determination condition in step S47 is satisfied, the processor 11A determines whether or not the determination condition in step S47 has been continuously satisfied for a predetermined time period of T21 or longer (step S48).

In this case, reference character T21 denotes a predetermined time period for determining whether or not a difference between the pressure value P2 measured by the water suction pressure gauge 16 and the pressure value Pi measured by the internal pressure sensor 37 is abnormal. The predetermined time period T21 is, for example, 3 seconds. Waiting for the predetermined time period T21 to elapse also enables chattering to be prevented.

When the determination condition in step S47 or step S48 is not satisfied, the processor 11A returns to the processing of step S46.

In addition, when the determination condition in step S46 is not satisfied, the processor 11A assigns F to the flag ref.Flag (step S49). This is because a change in the water suction condition at the time of creation of the reference necessitates recreating the reference.

When the determination condition in step S43 or step S44 is not satisfied or when the processing of step S49 is performed, the processor 11A returns to the processing of step S42.

### [When the Water Suction Pump is Stopped]

A case where the water suction pump 14 is operating has been described above. In contrast, when the water suction pump 14 is stopped, pressure at a distal end of the water suction system or, in other words, an internal pressure of the perfusion object 90 can be measured based on the pressure value P2 measured by the water suction pressure gauge 16 as long as the conduit of the water suction system is filled with water. Therefore, the pressure value P2 measured by the water suction pressure gauge 16 can be compared with the pressure value Pi measured by the internal pressure sensor 37.

When water suction Brake is turned on in step S42, the processor 11A assigns F to the flag ref Flag (step S50). In this case, the flag ref Flag is a flag indicating whether or not a pressure reference for calculating a relative change amount has been set.

In addition, the processor 11A makes a transition to the internal pressure sensor abnormality detection processing when the water suction pump 14 is being operated (step S51).

The processor 11A then determines whether or not the average value P2_ave of the pressure value P2 measured by the water suction pressure gauge 16 is larger than 0 (step S52).

In this case, the water suction pressure gauge 16 may measure negative pressure immediately after stopping water suction. Therefore, in step S52, it is confirmed that the average value P2_ave is at least equal to or higher than atmospheric pressure (in other words, the pressure value is positive).

At this point, when the average value P2_ave is larger than 0, the processor 11A determines whether or not |P2_ave−IRPressure|>dP is satisfied (step S53). As described above, the threshold dP is, for example, 30 (mmHg).

When the determination condition in step S53 is satisfied, the processor 11A determines whether or not the determination condition in step S53 has been continuously satisfied for the predetermined time period of T21 described above or longer (step S54).

When the determination condition is not satisfied in step S52, S53 or step S54, the processor 11A returns to the processing of step S42.

When the determination condition of step S48 or S54 is satisfied, the processor 11A assigns T to the flag IS_Error and, for example, issues a warning and stops the system (step S55).

As described above, according to the processing of steps S45 to S48 and S55, a determination of an internal pressure sensor abnormality is made when the water feed pump 13 and the water suction pump 14 are operated. In addition, according to the processing of steps S51 to S55, a determination of an internal pressure sensor abnormality is made when the water feed pump 13 is operated.

[Behavior after Abnormality] (Including Working of Abnormality Notifying Unit 11e5)

After determining an abnormality of the internal pressure sensor 37, the processor 11A notifies the user that an abnormality has occurred by an alarm, lighting an LED (light emitting diode), causing the LED to blink, displaying a message on a GUI (graphical user interface), or the like. In addition, the processor 11A may hide a pressure value that had originally been displayed, bracket a pressure value that had originally been displayed to indicate that the originally-displayed pressure value is a reference value, or the like.

Furthermore, since reliability of the pressure value measured by the internal pressure sensor 37 is low, the processor 11A may stop operations of the water feed pump 13 and the water suction pump 14.

After checking the notification, the user may continue a procedure while paying attention to a degree of bulging of, for example, a kidney that is the perfusion object 90. Alternatively, the user can also continue a procedure without using the water feed pump 13 and the water suction pump 14 by, in the same manner as a conventional procedure, feeding water by natural perfusion into the perfusion object 90 via the water suction channel 35 from the water feed source 51 such as a normal saline bag that is positioned higher than the perfusion object 90.

Once the processing in step S55 is performed, the processor 11A returns processing to main processing (not illustrated) or the like.

In addition, in order to confirm whether or not the warning in step S55 is correct, the user can perform only water feeding in a state where the water suction pump 14 has been stopped to check whether or not the pressure value P2 measured by the water suction pressure gauge 16 and the pressure value Pi measured by the internal pressure sensor 37 have really deviated from each other.

While an example of detecting an abnormality of the internal pressure sensor 37 by comparing the pressure value P2 measured by the water suction pressure gauge 16 and the pressure value Pi measured by the internal pressure sensor 37 with each other has been described in FIG. 16, the detection of an abnormality of the internal pressure sensor 37 is not limited thereto. For example, an abnormality of the internal pressure sensor 37 may be detected by comparing the pressure value P1 measured by the water feed pressure gauge 15 and the pressure value Pi measured by the internal pressure sensor 37 with each other.

Furthermore, an abnormality of the water suction pressure gauge 16 may be detected by comparing the pressure value P2 measured by the water suction pressure gauge 16 and the pressure value Pi measured by the internal pressure sensor 37 with each other. In addition, an abnormality of the water feed pressure gauge 15 may be detected by comparing the pressure value P1 measured by the water feed pressure gauge 15 and the pressure value Pi measured by the internal pressure sensor 37 with each other.

Note that for the purpose of detecting an abnormality of the internal pressure sensor 37, the water feed pressure gauge 15, or the water suction pressure gauge 16, the water feed flow rate meter 17 and the water suction flow rate meter 18 need not be provided. In addition, the valve 12 may or may not be provided.

The internal pressure sensor 37 which can be mounted to the distal end portion of the endoscope 30 or the like is small and expensive. By comparison, pressure sensors provided in the water feed pressure gauge 15 and the water suction pressure gauge 16 can be mounted inside the fluid control apparatus for endoscope 10 and are reusable, and are also relatively inexpensive.

Therefore, by performing processing such as that shown in FIG. 16, reliability of the internal pressure sensor 37 can be ensured by the pressure sensor provided in the water feed pressure gauge 15 or the water suction pressure gauge 16 and the endoscope fluid control system 1 can be configured so as to achieve downsizing of the distal end portion and cost reduction of the endoscope 30 and also to be suitable for single-use endoscopes.

[Estimation of Internal Pressure from Flow Rate]

In the description given above, utilizing a correlation between the pressure value measured by the water feed pressure gauge 15 or the water suction pressure gauge 16 and the pressure value measured by the internal pressure sensor 37, internal pressure of the perfusion object 90 is estimated from a pressure value measured by the water feed pressure gauge 15 or the water suction pressure gauge 16.

The internal pressure of the perfusion object 90 can also be estimated by a method to be described below.

As described above with reference to FIG. 5 and FIG. 6, in the endoscope fluid control system 1, a water hammer effect is generated by opening the valve 12 to the atmosphere for a short period of time during water suction to reverse a flow of a fluid inside the water suction conduit and snagging of a stone is removed. Internal pressure of the perfusion object 90 where the distal end of the water suction conduit is positioned can be estimated from a relationship between a flow rate of a backflow that is generated by the water hammer effect and a conduit resistance of the water suction system.

When estimating internal pressure from the flow rate of a backflow, although the water feed pressure gauge 15 and the water suction pressure gauge 16 need not be provided, the valve 12 (or a pinch valve or the like) must be provided.

First, a conduit resistance ΔP is calculated by equation (2) below.

$$Δ P = \frac{64}{Re} \times \frac{L}{D} \times \frac{ρ \times {v}^{2}}{2} \qquad ( 2 )$$

In equation (2), reference character Re denotes a Reynolds number, L denotes a length of a tube, D denotes an inner diameter of the tube, ρ denotes a density of a fluid, and v denotes flow velocity.

As shown in equation (3) below, a flow rate Q is given as a product of the flow velocity v and a cross-sectional area of the inner diameter of the tube.

$$Q = v \times {( \frac{D}{2} )}^{2} \times π \qquad ( 3 )$$

Equation (3) is transformed into equation (4) of the flow velocity v as

$$v = \frac{4 \times Q}{π \times {D}^{2}} \qquad ( 4 )$$

Substituting the flow velocity v of equation (4) into equation (2), equation (5) is obtained.

$$Δ P = \frac{64}{Re} \times ρ \times \frac{8 \times L \times {Q}^{2}}{{π}^{2} \times {D}^{5}} \qquad ( 5 )$$

In other words, once information such as the conduit length L, the tube inner diameter D, and the like is known, the conduit resistance ΔP is obtained from the flow rate Q according to equation (5).

Compared to the state shown in FIG. 5, a flow rate inside the water suction conduit is significantly lower in the state shown in FIG. 6. As a result, on a distal end side of the water suction conduit, the fluid attempting to flow in is pushed back due to a force of inertia and flows in reverse. However, when an entire water suction system is considered, positive pressure is applied to the distal end of the water suction system due to internal pressure of the perfusion object 90 (for example, intrarenal pressure) and a vicinity of the valve 12 is opened to the atmosphere and is close to atmospheric pressure. Therefore, albeit small, a flow of water is created in the water suction direction in the water suction conduit.

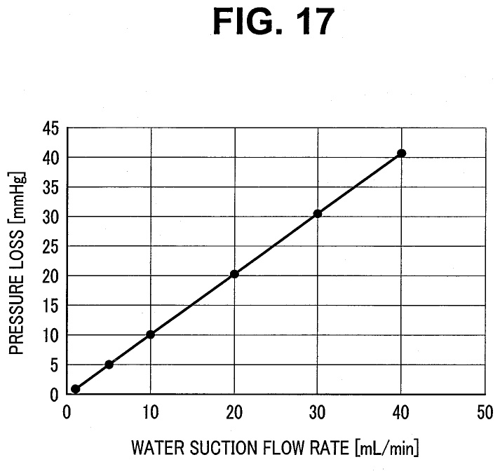

The flow rate of the water suction conduit at this point is to be determined based on a difference between discharge pressure (in this case, intrarenal pressure) and a pressure loss due to the conduit resistance ΔP and, as shown in FIG. 17, a relationship between the water suction flow rate and the internal pressure of the perfusion object 90 (intrarenal pressure) is a proportional relationship. In this case, FIG. 17 is a graph showing a relationship between the flow rate of the water suction conduit and pressure loss in the first embodiment.

In the graph shown in FIG. 17, while a constant of proportionality a of the internal pressure of the perfusion object 90 with respect to the water suction flow rate is close to 1, the constant of proportionality a changes according to a configuration of the water suction conduit. Therefore, the constant of proportionality a is desirably comprehended by acquiring, in advance, the relationship between the water suction flow rate and the internal pressure of the perfusion object 90 in the state shown in FIG. 6. When the relationship between the flow rate of the water suction conduit and pressure loss is obtained in advance, the internal pressure of the perfusion object 90 can be estimated from a flow rate measured by the water suction flow rate meter 18.

The relationship between flow rate and internal pressure is affected by the conduit resistance ΔP. Therefore, when the conduit resistance ΔP changes in accordance with an attitude of the conduit during actual use, an error is created in estimated internal pressure.

As is apparent from equation (5) (or equation (2)), when the inner diameter D of the tube increases, the conduit resistance ΔP decreases. In consideration thereof, in order to reduce the error in estimated internal pressure, the conduit is desirably made thicker in portions where it is possible to do so in order to reduce the conduit resistance ΔP.

In addition, while a similar idea is applied to the conduits inside the endoscope 30, there may be cases where a conduit inside the endoscope 30 cannot be realistically made thicker. In such a case, a slight change in the conduit resistance ΔP occurs in accordance with the attitude of the conduit during actual use. An internal pressure estimation error that is created due to such a change in the conduit resistance ΔP must also be taken into consideration. Furthermore, since the conduit resistance ΔP may also differ due to individual variability of the conduit and may similarly cause an internal pressure estimation error to be created, the internal pressure estimation error in this case must also be taken into consideration.

When there is a variation in conduits as described above, the processor 11A may be configured to read type information of the endoscope 30 obtained from a user setting, an EEPROM, an RFID, or the like to accommodate differences in the conduit resistance ΔP.

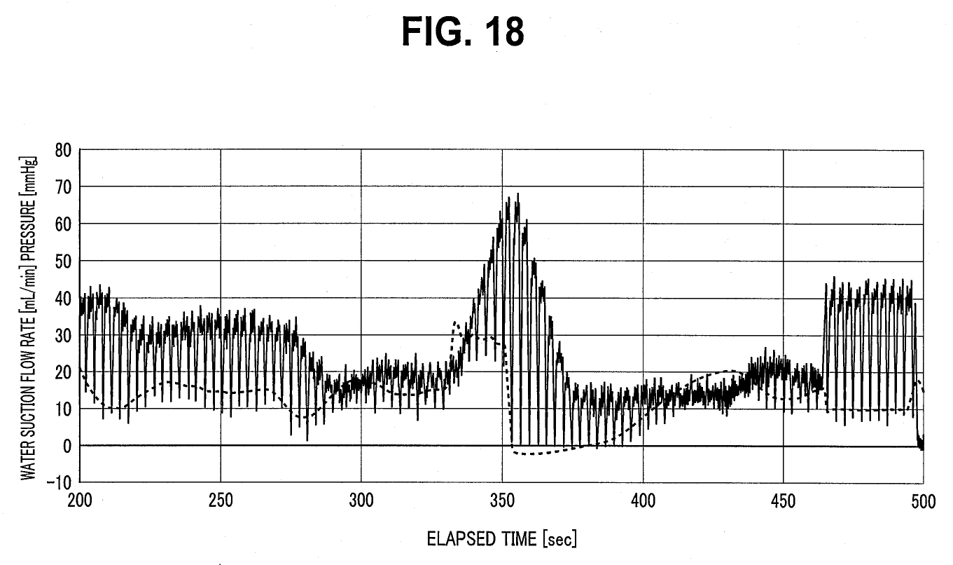

FIG. 18 is a graph showing a change with elapsed time of the water suction flow rate and the pressure value measured by the internal pressure sensor 37 according to the first embodiment. In FIG. 18, a solid line depicts a change in the water suction flow rate (in units of (mL/min)) measured by the water suction flow rate meter 18 and a dotted line depicts a change in pressure (in units of (mmHg)) inside the perfusion object 90 measured by the internal pressure sensor 37.

In the example shown in FIG. 18, an operation involving opening and closing the valve 12 at constant intervals and removing snagging of a stone by a water hammer effect is performed. Therefore, for example, numerical values represented by the solid line shown in FIG. 18 fluctuate up and down at intervals of around 3 seconds.

In the opening and closing intervals of the valve 12, a minimum value of the solid line represents a flow rate when the valve 12 is in the open state as shown in FIG. 6. By acquiring a flow rate at a minimum value of intervals on the solid line from the water suction flow rate meter 18 and referring to the graph shown in FIG. 17 which corresponds to the acquired minimum value, an estimated value of pressure inside the perfusion object 90 is obtained.

When the minimum value of the water suction flow rate in the intervals and the pressure value measured by the internal pressure sensor 37 are in a relationship of direct proportion as shown in FIG. 17, an estimated value of the pressure inside the perfusion object 90 may be obtained by dividing the minimum value of the water suction flow rate by the constant of proportionality a obtained from the relationship shown in FIG. 17.

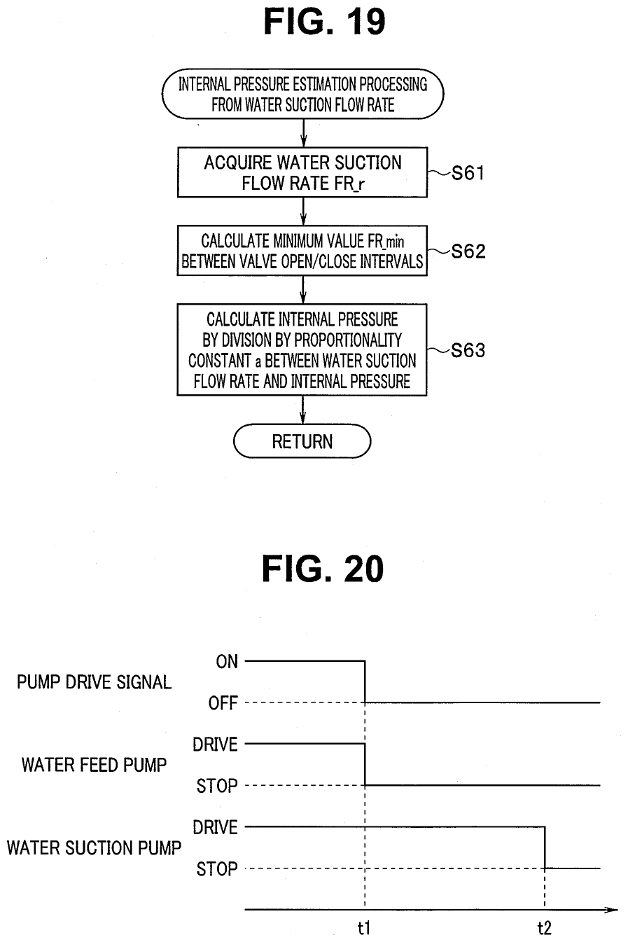

FIG. 19 is a flowchart showing processing of estimating the pressure inside the perfusion object 90 from a flow rate measured by the water suction flow rate meter 18 in the first embodiment. The processing shown in FIG. 19 is executed by the internal pressure estimating unit 11f of the controller 11.

When the processing shown in FIG. 19 is started, the processor 11A acquires a water suction flow rate value FR_r in real-time from the water suction flow rate meter 18 (step S61).

The processor 11A calculates a minimum value FR_min in the opening and closing intervals of the valve 12 among the acquired water suction flow rate value FR_r (step S62).

The processor 11A calculates an estimated value IRP_e of internal pressure by dividing the minimum value FR_min by the constant of proportionality a (step S63) and returns processing to main processing (not illustrated) or the like.

As described with reference to FIGS. 17 to 19, by estimating the pressure inside the perfusion object 90 from a flow rate measured by the water suction flow rate meter 18 when opening and closing the valve 12, there is no longer a need to mount the small and expensive internal pressure sensor 37 to the distal end portion of the endoscope 30. Accordingly, cost can be reduced and a diameter of the endoscope 30 can be further narrowed.

In addition, even when the internal pressure sensor 37 is mounted to the distal end portion of the endoscope 30, an abnormality of the internal pressure sensor 37 can be detected by comparing a pressure value measured by the internal pressure sensor 37 and a value estimated from a flow rate measured by the water suction flow rate meter 18 with each other (refer to FIG. 18).

### [Pump Stopping Method]

After performing water feeding and water suction using normal saline or the like and performing perfusion inside the perfusion object 90 for a certain time period, when a stone is discharged, the pump control unit 11g of the controller 11 is configured to stop the water feed pump 13 and the water suction pump 14. When stopping the pumps, pressure inside the perfusion object 90 must be taken into consideration.

For example, when inserting the insertion portion 31 into a narrow portion such as the ureter and performing perfusion inside a kidney being the perfusion object 90, a conduit may become narrow and conduit resistance may increase.

In this case, for example, when the conduit resistance on the water feeding side is larger than the conduit resistance on the water suction side, pressure may remain in the water feed system with the larger conduit resistance. As a result, even after stopping the water feed pump 13 and the water suction pump 14, water feeding to the perfusion object 90 ends up being performed and pressure inside the perfusion object 90 may possibly rise.

Hereinafter, methods of stopping the water feed pump 13 and the water suction pump 14 while suppressing a rise in pressure inside the perfusion object 90 will be described with reference to FIGS. 20 to 25. Control of each method is to be performed by the pump control unit 11g of the controller 11.

FIG. 20 is a timing chart showing a first example of a method of stopping the water feed pump 13 and the water suction pump 14 according to the first embodiment.

For example, a case is assumed where a pump drive signal is switched from on to off at a time point t1. As a result, due to control of the processor 11A, at the time point t1, drive of the water feed pump 13 is stopped but drive of the water suction pump 14 is continued.

Subsequently, at a time point t2 that is temporally posterior to the time point t1, due to control of the processor 11A, drive of the water suction pump 14 is stopped. While a time interval between the time point t1 and the time point t2 is, for example, 5 seconds, the time interval is not limited thereto.

For example, the time interval between the time point t1 and the time point t2 may be changed in accordance with at least one of the flow rate of the water feed pump 13 or the flow rate of the water suction pump 14 at the time point t1 at which the pump drive signal had been switched from on to off (or a time point immediately preceding the time point t1) (hereinafter, the time point t1 or the time point immediately preceding the time point t1 will be referred to as the time point t1 and the like).

More specifically, for example, the time interval may be changed at a rate of 1 (sec)/10 (mL/min). In this case, when the water feed pump 13 is being driven at 30 (mL/min) at the time point t1 and the like, the time interval between the time point t1 and the time point t2 may be set to 3 seconds.

In addition, when providing any of a configuration of measuring and a configuration of estimating the pressure inside the perfusion object 90 as described above, after stopping drive of the water feed pump 13 at the time point t1, the drive of the water suction pump 14 may be stopped at a time point where the internal pressure of the perfusion object 90 drops to or below a predetermined value (for example, 10 (mmHg)).

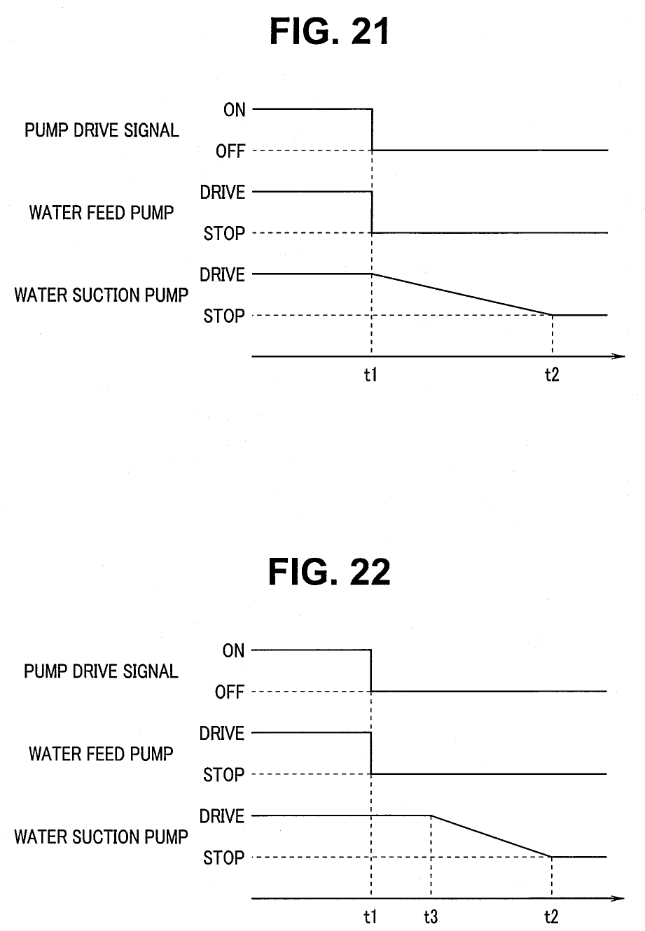

FIG. 21 is a timing chart showing a second example of a method of stopping the water feed pump 13 and the water suction pump 14 according to the first embodiment.

Due to control of the processor 11A, the water suction flow rate is gradually reduced at a constant rate (constant gradient) from the time point t1 at which the pump drive signal had been switched from on to off. A rate of the gradual reduction is, for example, −5 ((mL/min)/sec).

For example, when the water feed pump 13 is being driven at 30 (mL/min) at the time point t1 and the like, the time interval between the time point t1 and the time point t2 is 6 seconds.

Note that the rate of gradual reduction may be changed in accordance with at least one of the flow rate of the water feed pump 13 or the flow rate of the water suction pump 14 at the time point t1 and the like.

FIG. 22 is a timing chart showing a third example of a method of stopping the water feed pump 13 and the water suction pump 14 according to the first embodiment.

The third example shown in FIG. 22 is a method that combines the first example shown in FIG. 20 with the second example shown in FIG. 21.

In other words, due to control of the processor 11A, at the time point t1 at which the pump drive signal had been switched from on to off, drive of the water feed pump 13 is stopped but drive of the water suction pump 14 is continued.

Subsequently, at a time point t3 that is temporally posterior to the time point t1 and temporally anterior to the time point t2, due to control of the processor 11A, the water suction flow rate is gradually reduced by a constant rate (constant gradient).

The rate of gradual reduction at this point is a rate that causes the flow rate of the water suction pump 14 at the time point t3 to become 0 at the time point t2.

Note that a time interval between the time point t1 and the time point t3 may be changed in accordance with at least one of the flow rate of the water feed pump 13 or the flow rate of the water suction pump 14 at the time point t1 and the like.

In a similar manner, a time interval between the time point t3 and the time point t2 may be changed in accordance with at least one of the flow rate of the water feed pump 13 or the flow rate of the water suction pump 14 at the time point t1 and the like.

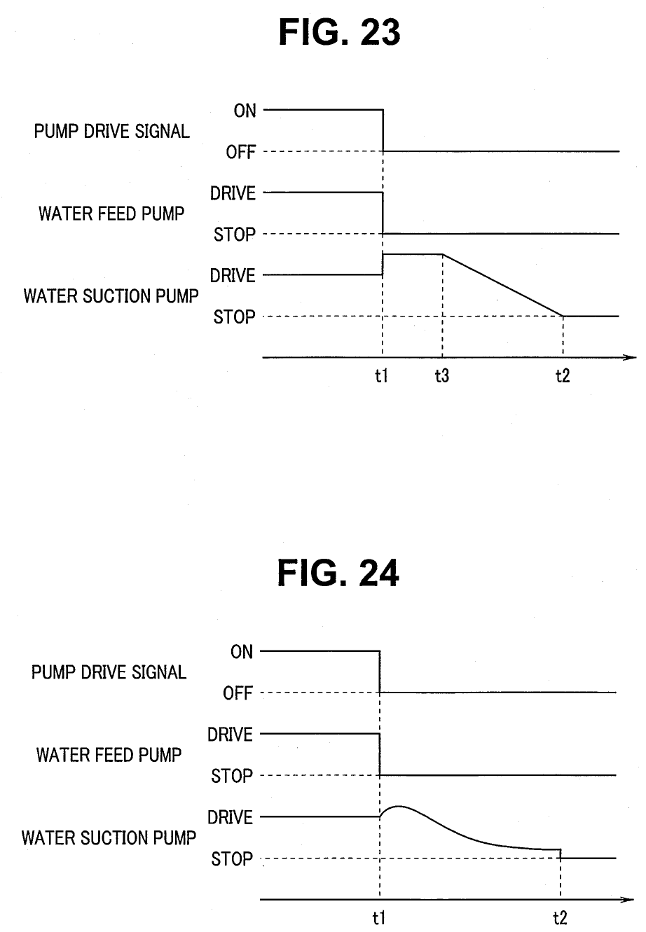

FIG. 23 is a timing chart showing a fourth example of a method of stopping the water feed pump 13 and the water suction pump 14 according to the first embodiment.

Due to control of the processor 11A, at the time point t1 at which the pump drive signal had been switched from on to off, drive of the water feed pump 13 is stopped but drive of the water suction pump 14 is continued while increasing the water suction flow rate by a constant amount.

Subsequently, at the time point t3 that is temporally posterior to the time point t1 and temporally anterior to the time point t2, due to control of the processor 11A, the water suction flow rate is gradually reduced by a constant rate (constant gradient).

In this case, a difference between the water suction flow rate from the time point t1 to the time point t3 and the water suction flow rate prior to the time point t1 or, in other words, an increase in the water suction flow rate is, for example, 10 (mL/min). In addition, a time interval between the time point t1 and the time point t3 is, for example, 2 seconds.

Note that the increase in the water suction flow rate may be changed in accordance with at least one of the flow rate of the water feed pump 13 or the flow rate of the water suction pump 14 at the time point t1 and the like.

In addition, the time interval between the time point t1 and the time point t3 may be changed in accordance with at least one of the flow rate of the water feed pump 13 or the flow rate of the water suction pump 14 at the time point t1 and the like.

In a similar manner, a time interval between the time point t3 and the time point t2 may be changed in accordance with at least one of the flow rate of the water feed pump 13 or the flow rate of the water suction pump 14 at the time point t1 and the like.

While the water suction flow rate is gradually reduced from the time point t3 in the example shown in FIG. 23, the water suction pump 14 may be stopped at the time point t2 without gradually reducing the water suction flow rate. The time interval between the time point t1 and the time point t2 in this case may be changed in accordance with at least one of the flow rate of the water feed pump 13 or the flow rate of the water suction pump 14 at the time point t1 and the like.

The internal pressure of the perfusion object 90 is most likely to rise immediately after stopping the water feed pump 13. Therefore, a rise in the internal pressure of the perfusion object 90 can be more suitably suppressed by increasing the water suction flow rate of the water suction pump 14 immediately after stopping the water feed pump 13.

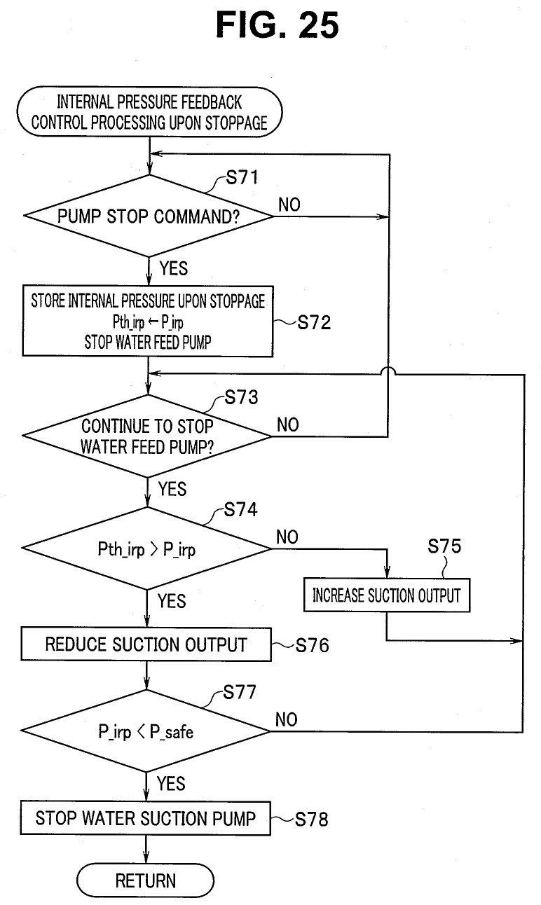

FIG. 24 is a timing chart showing a fifth example of a method of stopping the water feed pump 13 and the water suction pump 14 according to the first embodiment. FIG. 25 is a flowchart showing an example of internal pressure feedback control processing upon stopping the water feed pump 13 and the water suction pump 14 according to the first embodiment. The processing shown in FIG. 25 is executed by the pump control unit 11g of the controller 11.

When performing the processing shown in FIG. 25, a case of providing any configuration for measuring and estimating pressure inside the perfusion object 90 as described above is assumed. Hereinafter, an example of providing the internal pressure sensor 37 will be described.

When the processing shown in FIG. 25 is started, the processor 11A determines whether or not the processor 11A has received a pump stop command transmitted from an input apparatus such as an operation switch in accordance with, for example, an operation by the user (step S71).

At this point, when the pump stop command has not been received, the processor 11A stands by to receive the pump stop command.

In addition, when the pump stop command is received in step S71, the processor 11A records a pressure value P_irp inside the perfusion object 90 measured by the internal pressure sensor 37 as a threshold Pth_irp of the internal pressure. Furthermore, the processor 11A switches a pump drive signal from on to off at the time point t1 shown in FIG. 24 and stops the water feed pump 13 (step S72).

Subsequently, the processor 11A determines whether or not the stoppage of the water feed pump 13 is to be continued (step S73). At this point, when the stoppage of the water feed pump 13 is not continued due to, for example, the pump stop command being canceled or the like, the processor 11A returns to the processing of step S71 and stands by to receive a new pump stop command.

In addition, when the stoppage of the water feed pump 13 is continued in step S73, the processor 11A determines whether or not the pressure value P_irp inside the perfusion object 90 measured by the internal pressure sensor 37 is smaller than the threshold Pth_irp of the internal pressure (step S74).

When the pressure value P_irp is equal to or higher than the threshold Pth_irp of the internal pressure, the processor 11A increases the water suction flow rate of the water suction pump 14 (increases suction output) (step S75) and returns to the processing of step S73.

On the other hand, when the pressure value P_irp is lower than the threshold Pth_irp of the internal pressure in step S74, the processor 11A reduces the water suction flow rate of the water suction pump 14 (reduces suction output) (step S76).

In addition, the processor 11A determines whether or not the pressure value P_irp is lower than a predetermined internal pressure value P_safe (step S77). In this case, the predetermined internal pressure value P_safe is pressure at which it is determined that the water suction pump 14 can be stopped safely (more specifically, pressure at which even when the water suction pump 14 is stopped, pressure inside the perfusion object 90 does not exceed predetermined pressure (for example, the threshold Pth_irp of the internal pressure)).

In step S77, when the pressure value P_irp is equal to or higher than the predetermined internal pressure value P_safe, the processor 11A returns to the processing of step S73.

In addition, in step S77, when the pressure value P_irp is lower than the predetermined internal pressure value P_safe (which corresponds to the time point t2 in FIG. 24), the processor 11A stops the water suction pump 14 (step S78) and returns processing to main processing (not illustrated) or the like.

When the processing described above is performed, a water suction operation of the water suction pump 14 such as that shown in FIG. 24 is performed. However, since the manner in which the water suction pump 14 performs the water suction operation changes depending on states of the endoscope fluid control system 1 and the perfusion object 90 due to feedback control, the water suction operation shown in FIG. 24 merely represents an example.

Note that feedback control is not limited to an example of performing feedback control until falling below the predetermined internal pressure value P_safe as shown in FIG. 25. For example, feedback control such as PID (proportional-integral-differential) control which controls an input value based on three elements, namely, a deviation between an output value and a target value, an integral thereof, and a differential thereof may be adopted upon pump stoppage.

Even performing processing such as that shown in FIG. 24 and FIG. 25 enables a rise in the internal pressure of the perfusion object 90 when stopping the water feed pump 13 and the water suction pump 14 to be suitably suppressed.

Among the methods of stopping the water feed pump 13 and the water suction pump 14 described in the first to fifth examples, a method that functions most suitably with respect to the endoscope fluid control system 1 may be selected. In addition, when more or less the same function can be fulfilled, a simpler control method may be selected.

As described above, in the fluid control apparatus for endoscope 10 including the water feed pump 13 configured to feed a fluid into the perfusion object 90, the water suction pump 14 configured to recover the fed fluid, and the pump control unit 11g configured to control the water feed pump 13 and the water suction pump 14, when the pump control unit 11g receives an instruction from the user to stop the water feed pump 13 and the water suction pump 14, the pump control unit 11g is configured to stop the water suction pump 14 after a delay from the water feed pump 13. Accordingly, a rise in the internal pressure of the perfusion object 90 can be suppressed.

According to the first embodiment, in means for measuring pressure inside the perfusion object 90, the water feed pressure gauge 15 configured to measure pressure of a fluid inside a water feed conduit or the water suction pressure gauge 16 configured to measure pressure of a fluid inside a water suction conduit can be used to monitor whether or not the internal pressure sensor 37 configured to measure pressure inside the perfusion object 90 is operating normally.

In addition, with the endoscope fluid control system, the fluid control apparatus for endoscope, the control method of the endoscope fluid control system, and the control method of the fluid control apparatus for endoscope according to the first embodiment, internal pressure of a subject can be comprehended with low cost and reduced space.

While a case where the present invention is the fluid control apparatus for endoscope 10 and the endoscope fluid control system 1 including the fluid control apparatus for endoscope 10 has been heretofore mainly described, the present invention is not limited thereto. For example, the present invention may be a control method of the fluid control apparatus for endoscope 10 or a control method of the endoscope fluid control system 1. In addition, the present invention may be a computer program that causes a computer to perform processing similar to the fluid control apparatus for endoscope 10 or the endoscope fluid control system 1. Furthermore, the present invention may be a computer-readable non-transitory recording medium or the like which records the computer program.

In this case, examples of recording media that record computer program products include portable recording media such as a flexible disk, a CD-ROM (compact disc read only memory), a DVD (digital versatile disc), a USB (universal serial bus) flash drive, and a flash memory such as an SD memory card and recording media such as an HDD (hard disk drive) or an SSD (solid state drive). The recording media are not limited to recording an entirety of a computer program and may only record a part of the computer program. In addition, an entirety or a part of a computer program may be distributed or provided via a communication network. By installing a computer program on a computer from a recording medium or downloading a computer program via a communication network and installing the computer program on a computer, a user can have the computer read the computer program and execute all of or a part of operations to execute operations of the apparatuses described above.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An endoscope fluid control system, comprising:
an endoscope including a first water suction conduit;
a second water suction conduit detachably connected to the first water suction conduit;
a pump connected to the second water suction conduit;
a pressure gauge installed on the second water suction conduit;
a memory configured to record correlation data which indicates a correlation between a pressure value indicated by the pressure gauge and an output value of the pump and which is set for each type of the endoscope; and
a processor, wherein
the processor is configured to:
specify correlation data corresponding to a type of the endoscope the first water suction conduit of which is connected to the second water suction conduit; and
monitor an operation of the pump based on the specified correlation data.

2. The endoscope fluid control system according to claim 1, wherein
the memory is configured to record, at least for each type of the endoscope, first minimum correlation data that is constructed based on a first pressure value indicated by the pressure gauge and an output value of the pump corresponding to the first pressure value, and
the processor is configured to:
construct second minimum correlation data based on a second pressure value measured by the pressure gauge and the output value of the pump corresponding to the second pressure value when the first water suction conduit is connected to the second water suction conduit and the pump is operated;
specify correlation data which approximates combined data of the first minimum correlation data and the second minimum correlation data from among the correlation data set for each type of the endoscope which is recorded in the memory; and
monitor an operation of the pump based on the specified correlation data.

3. The endoscope fluid control system according to claim 1, wherein
the endoscope includes an internal pressure sensor in a distal end portion of an insertion portion, and
the processor is configured to:
calculate a difference between a first relative change amount of a pressure value measured by the pressure gauge from a first reference value and a second relative change amount of a pressure value measured by the internal pressure sensor from a second reference value; and
determine that the internal pressure sensor is abnormal when the difference exceeds a threshold.

4. The endoscope fluid control system according to claim 1, wherein
the pressure gauge is installed on the second water suction conduit between the pump and the first water suction conduit.

5. A control method of an endoscope fluid control system, comprising:
in a state where a first water suction conduit included in an endoscope and a second water suction conduit connected to a pump are connected to each other,
specifying, from correlation data which indicates a correlation between a pressure value indicated by a pressure gauge installed on the second water suction conduit and an output value of the pump, which is set for each type of the endoscope, and which is recorded in a memory, correlation data corresponding to a type of the endoscope the first water suction conduit of which is connected to the second water suction conduit; and
monitoring an operation of the pump based on the specified correlation data.

6. The control method of an endoscope fluid control system according to claim 5, wherein
the memory is configured to record, at least for each type of the endoscope, first minimum correlation data that is constructed based on a first pressure value indicated by the pressure gauge and an output value of the pump corresponding to the first pressure value, the control method of an endoscope fluid control system further comprising:
constructing second minimum correlation data based on a second pressure value measured by the pressure gauge and the output value of the pump corresponding to the second pressure value when the first water suction conduit is connected to the second water suction conduit and the pump is operated;
specifying correlation data which approximates combined data of the first minimum correlation data and the second minimum correlation data from among the correlation data set for each type of the endoscope which is recorded in the memory; and
monitoring an operation of the pump based on the specified correlation data.

7. A fluid control apparatus for endoscope, comprising:
a second water suction conduit detachably connected to a first water suction conduit included in an endoscope;
a pump connected to the second water suction conduit;
a pressure gauge installed on the second water suction conduit;
a memory configured to record correlation data which indicates a correlation between a pressure value indicated by the pressure gauge and an output value of the pump and which is set for each type of the endoscope; and
a processor, wherein
the processor is configured to:
specify correlation data corresponding to a type of the endoscope the first water suction conduit of which is connected to the second water suction conduit; and
monitor an operation of the pump based on the specified correlation data.

8. The fluid control apparatus for endoscope according to claim 7, wherein
the memory is configured to record, at least for each type of the endoscope, first minimum correlation data that is constructed based on a first pressure value indicated by the pressure gauge and an output value of the pump corresponding to the first pressure value, and
the processor is configured to:
construct second minimum correlation data based on a second pressure value measured by the pressure gauge and the output value of the pump corresponding to the second pressure value when the first water suction conduit is connected to the second water suction conduit and the pump is operated;
specify correlation data which approximates combined data of the first minimum correlation data and the second minimum correlation data from among the correlation data set for each type of the endoscope which is recorded in the memory; and
monitor an operation of the pump based on the specified correlation data.

9. The fluid control apparatus for endoscope according to claim 7, wherein
the processor is configured to select, based on information transmitted from an input apparatus in accordance with an operation by a user, one type of correlation data from among correlation data set for each type of the endoscope which is recorded in the memory.

10. The fluid control apparatus for endoscope according to claim 7, wherein
the processor is configured to select, in accordance with a type of the endoscope the first water suction conduit of which is connected to the second water suction conduit, one type of correlation data from among correlation data set for each type of the endoscope which is recorded in the memory.

11. The fluid control apparatus for endoscope according to claim 7, wherein
the pressure gauge is installed on the second water suction conduit between the pump and the first water suction conduit.

12. A fluid control apparatus for endoscope, comprising:
a second water suction conduit detachably connected to a first water suction conduit included in an endoscope;
a pump connected to the second water suction conduit;
a pressure gauge installed on the second water suction conduit;
a memory configured to record first minimum correlation data which indicates a correlation between a pressure value indicated by the pressure gauge and an output value of the pump and which is constructed based on a first pressure value indicated by the pressure gauge and an output value of the pump corresponding to the first pressure value; and
a processor, wherein
the processor is configured to:
construct second minimum correlation data based on a second pressure value measured by the pressure gauge and the output value of the pump corresponding to the second pressure value when the first water suction conduit is connected to the second water suction conduit and the pump is operated;
construct correlation data based on a combination of the first minimum correlation data and the second minimum correlation data; and
monitor an operation of the pump based on the constructed correlation data.

13. The fluid control apparatus for endoscope according to claim 12, wherein
the memory is configured to record a plurality of types of the first minimum correlation data the first pressure value indicated by the pressure gauge or the output value of the pump of which differ from each other, and
the processor is configured to select one type of first minimum correlation data from among the plurality of types of first minimum correlation data recorded in the memory.

14. The fluid control apparatus for endoscope according to claim 13, wherein
the processor is configured to select, in accordance with a type of the endoscope the first water suction conduit of which is connected to the second water suction conduit, one type of first minimum correlation data from among the plurality of types of first minimum correlation data recorded in the memory.

15. The fluid control apparatus for endoscope according to claim 13, wherein
the processor is configured to select, based on information transmitted from an input apparatus in accordance with an operation by a user, one type of first minimum correlation data from among the plurality of types of first minimum correlation data recorded in the memory.

16. The fluid control apparatus for endoscope according to claim 14, wherein
the processor is configured to:
acquire type information from the endoscope the first water suction conduit of which is connected to the second water suction conduit; and
determine the type of the endoscope based on the type information.

17. A control method of a fluid control apparatus for endoscope, comprising:
operating a pump in a state where a first water suction conduit included in an endoscope and a second water suction conduit connected to the pump are connected to each other;
reading, from a memory, first minimum correlation data which indicates a correlation between a pressure value indicated by a pressure gauge installed on the second water suction conduit and an output value of the pump and which is constructed based on a first pressure value indicated by the pressure gauge and an output value of the pump corresponding to the first pressure value;
constructing second minimum correlation data based on a second pressure value measured by the pressure gauge and an output value of the pump corresponding to the second pressure value;
constructing correlation data based on a combination of the first minimum correlation data and the second minimum correlation data; and
monitoring an operation of the pump based on the constructed correlation data.

18. The control method of a fluid control apparatus for endoscope according to claim 17, wherein
the memory is configured to record a plurality of types of the first minimum correlation data the first pressure value indicated by the pressure gauge or the output value of the pump of which differ from each other, and
the control method of a fluid control apparatus for endoscope comprises selecting one type of first minimum correlation data from among the plurality of types of first minimum correlation data recorded in the memory.

19. The control method of a fluid control apparatus for endoscope according to claim 18, comprising,
selecting, in accordance with a type of the endoscope the first water suction conduit of which is connected to the second water suction conduit, one type of first minimum correlation data from among the plurality of types of first minimum correlation data recorded in the memory.

20. The control method of a fluid control apparatus for endoscope according to claim 18, comprising,
selecting, based on information transmitted from an input apparatus in accordance with an operation by a user, one type of first minimum correlation data from among the plurality of types of first minimum correlation data recorded in the memory.

21. The control method of a fluid control apparatus for endoscope according to claim 19, comprising:
acquiring type information from the endoscope the first water suction conduit of which is connected to the second water suction conduit; and
determining the type of the endoscope based on the type information.
